(19) United States
(12) Patent Application Publication
(10) Pub. No.: US 2006/0229513 A1
(43) Pub. Date: Oct. 12, 2006

(54) DIAGNOSTIC IMAGING SYSTEM AND IMAGE PROCESSING SYSTEM

(75) Inventor: Satoshi WAKAI, Nasushiobara-Shi (JP)

Correspondence Address: C. IRVIN MCCLELLAND;OBLON, SPIVAK, MCCLELLAND, MAIER & NEUSTADT, P.C., 1940 DUKE STREET, ALEXANDRIA, VA, 22314, US

(73) Assignees: KABUSHIKI KAISHA TOSHIBA, Minato-Ku (JP); TOSHIBA MEDICAL SYSTEMS, Otawara-Shi (JP)

(21) Appl. No.: 11/278,764

(22) Filed: Apr. 5, 2006

(30) Foreign Application Priority Data
Apr. 6, 2005 (JP) 2005-110042

(51) Int. Cl. A61B 5/05; G06K 9/00

(52) U.S. Cl. 600/407; 382/128

(57) ABSTRACT

A functional image analyzing unit in an image processing system extracts an active region from the functional information data, serving as volume data, and a display-priority determining unit determines the display-priority on the basis of a volume of the active region or a voxel value of the active region. An image data fusing unit fuses functional image data and morphological image data to create fused-image data, and an image creating unit receives the fused-image data and sequentially creates three-dimensional image data in accordance with the display-priority. A display control unit allows a plurality of pieces of the three-dimensional image data to sequentially be displayed on a display. Herewith, it is possible to efficiently make a diagnosis and a diagnostic reading by a user, because a time for searching a targeted active region by the user can be reduced.

### BACKGROUND OF THE INVENTION

1. Field of the Invention

The present invention relates to a technology, with which, an image indicating a region for observation is created and displayed on the basis of a morphological image captured by an X-ray computerized tomography (X-ray CT) apparatus, a magnetic resonance imaging (MRI) apparatus, or an ultrasonic diagnostic apparatus and a functional image captured by a nuclear medicine diagnostic apparatus or a functional-magnetic resonance imaging (f-MRI) apparatus. In particular, the present invention relates to a diagnostic imaging system and an image processing system that roughly specify the position of the lesion with the functional image and finely observes the position and the shape of the lesion on the morphological image.

2. Description of the Related Art

In general, the clinical diagnosis includes a morphological diagnosis and a functional diagnosis. Importantly in view of the clinical diagnosis, it is determined whether or not a disease causes the tissue or the organ to normally function. With diseases, the abnormality of the function metastasizes, thereby changing an anatomical morphology of the tissue. An MRI apparatus, an X-ray CT apparatus, or an ultrasonic diagnostic apparatus is used for the morphological diagnosis. For example, with the X-ray CT apparatus, X rays are extracorporeally emitted, and a tomographic image is reconstructed on the basis of a value obtained by measuring the transmitted X-rays with a detector.

At the same time, there is a method said as a nuclear medicine diagnosis. As for the nuclear medicine diagnosis, a feature that a radio isotope (RI) or a labeled compound thereof is selectively absorbed to a specific tissue or organ in the living body is used, γ rays emitted from the RI are extracorporeally measured, and the dose distribution of RI as an image is diagnosed. The nuclear medicine diagnosis enables not only the morphological diagnosis but also the functional diagnosis of an early state of the lesion. A nuclear medicine diagnostic apparatus includes a positron emission computed tomograpy (PET) apparatus and a single photon emission computed tomograpy (SPECT) apparatus. In addition to the nuclear medicine diagnostic apparatus, an f-MRI apparatus is used, particularly, for the functional diagnosis of the brain.

Conventionally, when a user mainly observes a functional active region of a tumor by using a three-dimensional image as a medical image, an operation for partly preventing an image display operation is performed by clipping processing, image selecting processing and so on, thereby observing an image of the targeted tumor.

Further, the inside of a tubular tissue, such as the blood vessel, the intestine, and the bronchi, is observed with so-called display operation via virtual endoscopy based on image data collected by the X-ray CT apparatus or the like. With the display operation via the virtual endoscopy, e.g., three-dimensional image data of a morphological image is created and the created three-dimensional image data is displayed as a three-dimensional image.

However, with the display operation via the virtual endoscopy using the three-dimensional image data having only the morphological image, although the shape, size, and position, of the active region can manually be checked, the state of the active region cannot manually be checked.

Further, with the conventional technology, although it is possible to display the three-dimensional image obtained by superimposing the morphological image and the functional image, an operator, e.g., a doctor needs to search for the position of the active region, such as the tumor, by manually performing the operation including the clipping processing and the image selection. Thus, the observation of the targeted active region consumes time and labor, an image of the active region is not easily displayed, and the interpretation and diagnosis are not efficient.

Furthermore, even if obtaining the targeted image, the display format of the image is insufficient, e.g., the viewpoint with the active region for observation as center is not automatically determined. Therefore, diagnostic information is not presented sufficiently to the doctor, etc. and this does not enable the efficient diagnosis.

In addition, the positions and the states of all active regions are not grasped before executing the display operation via the virtual endoscopy and it is necessary to search the active region by executing the display operation via the virtual endoscopy. Especially, with the display operation via the virtual endoscopy using the three-dimensional image data containing only the morphological image, all branches of the tubular organ need to be completely searched. In this case, the search of the active region consumes labor and time and the efficient interpretation and diagnosis are not possible. Further, there is a danger of the miss of the active region.

### SUMMARY OF THE INVENTION

The present invention has taken into consideration the above-described problems, and it is an object of the present invention to provide a diagnostic imaging system and an image processing system such that it efficiently make a diagnosis and a diagnostic reading by a user, by reducing a time for searching a targeted active region by the user.

As mentioned in claim 1 to solve the above-described problems, the present invention provides the diagnostic imaging system, comprising: an active region extracting unit for obtaining functional information data indicating functional information of the object, and for extracting an active region from the functional information data; an image data fusing unit for fusing the active region extracted by the active region extracting unit and the image of the inside of the tubular tissue; and a display control unit for allowing the image fused by the image data fusing unit to be displayed.

As mentioned in claim 6 to solve the above-described problems, the present invention provides the diagnostic imaging system, comprising: an active region extracting unit for obtaining functional information data indicating functional information of the object, and for extracting an active region from the functional information data; an image data fusing unit for fusing the active region extracted by the active region extracting unit and an image indicating a path of the tubular tissue; and a display control unit for allowing the image fused by the image data fusing unit to be displayed.

As mentioned in claim 9 to solve the above-described problems, the present invention provides the diagnostic imaging system, comprising: an image data fusing unit for fusing functional image data, serving as volume data collected by capturing an object, and morphological image data, serving as the volume data, to create fused-image data, serving as the volume data; an active region extracting unit for extracting the active region from the functional image data; an image creating unit for creating three-dimensional image data obtained by superimposing the functional image and the morphological image along a specific line-of-sight direction relative to the active region, on the basis of the fused-image data; and a display control unit for allowing the three-dimensional image data to be displayed as a three-dimensional image.

As mentioned in claim 10 to solve the above-described problems, the present invention provides the image processing system, comprising: an active region extracting unit for obtaining functional information data indicating functional information of the object, and for extracting an active region from the functional information data; an image data fusing unit for fusing the active region extracted by the active region extracting unit and the image of the inside of the tubular tissue; and a display control unit for allowing the image fused by the image data fusing unit to be displayed.

As mentioned in claim 15 to solve the above-described problems, the present invention provides the image processing system, comprising: an active region extracting unit for obtaining functional information data indicating functional information of the object, and for extracting an active region from the functional information data; an image data fusing unit for fusing the active region extracted by the active region extracting unit and an image indicating a path of the tubular tissue; and a display control unit for allowing the image fused by the image data fusing unit to be displayed.

As mentioned in claim 18 to solve the above-described problems, the present invention provides the image processing system, comprising: an image data fusing unit for fusing functional image data, serving as volume data collected by capturing an object, and morphological image data, serving as the volume data, to create fused-image data, serving as the volume data; an active region extracting unit for extracting the active region from the functional image data; an image creating unit for creating three-dimensional image data obtained by superimposing the functional image and the morphological image along a specific line-of-sight direction relative to the active region, on the basis of the fused-image data; and a display control unit for allowing the three-dimensional image data to be displayed as a three-dimensional image.

Therefore, according to the present invention to provide the diagnostic imaging system and the image processing system, it is possible to efficiently make a diagnosis and a diagnostic reading by a user, because a time for searching a targeted active region by the user can be reduced.

### BRIEF DESCRIPTION OF THE DRAWINGS

In the accompanying drawings:

FIG. 1 is a block diagram showing a structure of a diagnostic imaging system and an image processing system according to a first embodiment of the present invention;

FIG. 2 is a drawing for explaining a parallel projection in a volume rendering;

FIG. 3 is a drawing for explaining a perspective projection in a volume rendering;

FIG. 4 is a flowchart for an operation of a diagnostic imaging system and an image processing system according to a first embodiment of the present invention;

FIG. 5 is a drawing for explaining an extracting processing of an active region from a functional image data, serving as a volume data;

FIG. 6 is a drawing for explaining a fusing processing of a morphological image data and a functional image data;

FIG. 7 is a drawing showing one example of a three-dimensional image obtained from a three-dimensional image data via a virtual endoscopy;

FIG. 8 is a drawing showing one example of a three-dimensional image obtained from a three-dimensional image data indicating an appearance of a tubular region;

FIG. 9 is a drawing for explaining how to determine a line-of-sight direction;

FIG. 10 is a drawing for explaining how to obtain a line-of-sight direction from an active region;

FIG. 11 is a drawing showing one example of a monitor screen of a display device;

FIG. 12 is a drawing showing another example of the monitor screen of the display device;

FIG. 13 is a block diagram showing a structure of a diagnostic imaging system and an image processing system according to a second embodiment of the present invention;

FIG. 14 is a flowchart for explaining an operation of a diagnostic imaging system and an image processing system according to a second embodiment of the present invention;

FIG. 15 is a drawing for explaining a determining processing of a display-priority about a three-dimensional image of an active region;

FIG. 16 is a drawing for explaining a point of view movement;

FIG. 17 is a drawing showing one example of a monitor screen of a display device;

FIG. 18 is a block diagram showing a structure of a diagnostic imaging system and an image processing system according to a third embodiment of the present invention;

FIG. 19 is a flowchart showing an operation of a diagnostic imaging system and an image processing system according to a third embodiment of the present invention;

FIG. 20 is a drawing for explaining a determining processing of a display-priority about a path;

FIG. 21 is a drawing showing a path displayed via a virtual endoscopy;

FIG. 22 is a drawing showing a path displayed via a virtual endoscopy;

FIG. 23 is a drawing showing one example of a monitor screen of a display device; and

FIG. 24 is a drawing showing another example of the monitor screen of the display device.

### DESCRIPTION OF THE PREFERRED EMBODIMENTS

A description is given of a diagnostic imaging system and an image processing system according to embodiments of the present invention with reference to the accompanied drawings.

### First Embodiment

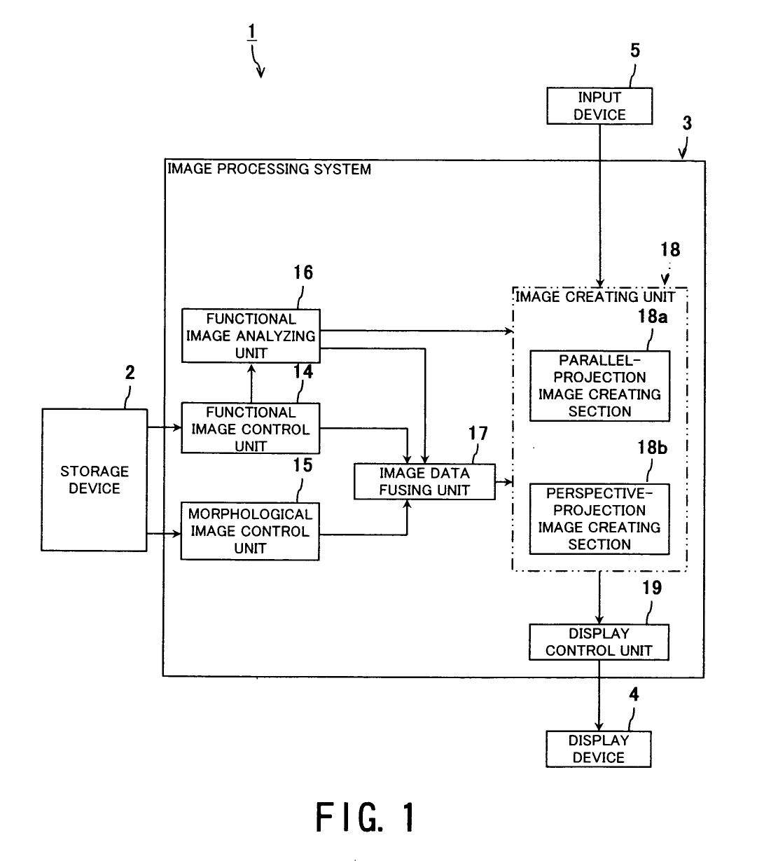

FIG. 1 is a block diagram showing a structure of a diagnostic imaging system and an image processing system according to a first embodiment of the present invention.

Referring to FIG. 1, a diagnostic imaging system 1 is shown, and the diagnostic imaging system 1 comprises a storage device 2, an image processing system 3, a display device 4, and an input device 5. Note that the diagnostic imaging system 1 includes therein, the storage device 2, the image processing system 3, the display device 4, and the input device 5 as shown in FIG. 1, however, the present invention is not limited to this structure, and the diagnostic imaging system 1 may externally have a part or all of the storage device 2, the image processing system 3, the display device 4, and the input device 5.

The storage device 2 comprises a hard disk, a memory and so on, and mainly stores functional image data and morphological image data. Specifically, the storage device 2 stores the functional image data, serving as two-dimensional image data, collected by a nuclear medicine diagnosis (e.g., PET apparatus or SPECT apparatus) or an f-MRI apparatus. Further, the storage device 2 stores the morphological image data (tomographic image data), serving as two-dimensional image data, collected by an X-ray CT apparatus, an MRI apparatus, or an ultrasonic diagnostic apparatus.

The image processing system 3 comprises a functional image control unit 14, a morphological image control unit 15, a functional image analyzing unit 16, an image data fusing unit 17, an image creating unit 18, and a display control unit 19. Note that the units 14 to 19 in the image processing system 3 may be provided as hardware of the image processing system 3 and, alternatively, may function as software.

The functional image control unit 14 in the image processing system 3 reads a plurality of pieces of the functional image data, serving as two-dimensional data, from the storage device 2 and interpolates the read image data, thereby creating the functional image data, serving as volume data (voxel data) expressed on three-dimensional real space. The functional image control unit 14 outputs the functional image data, serving as the volume data, to the functional image analyzing unit 16 and the image data fusing unit 17. Although not shown, the functional image control unit 14 can output the functional image data, serving as the volume data, to the image creating unit 18.

The morphological image control unit 15 reads a plurality of pieces of two-dimensional morphological image data, from the storage device 2, and interpolates the read image data, thereby creating the morphological image data, serving as the volume data expressed on three-dimensional real space. The morphological image control unit 15 outputs the morphological image data, serving as the volume data, to the image data fusing unit 17. Although not shown, the morphological image control unit 15 can output the morphological image data, serving as the volume data, to the image creating unit 18.

Note that when the diagnostic imaging system 1 can directly collect the volume data, the storage device 2 stores the functional image data and the morphological image data, serving as the volume data. When the storage device 2 stores the volume data, the functional image control unit 14 reads the volume data from the storage device 2, and outputs the volume data to the functional image analyzing unit 16 and the image data fusing unit 17. On the other hand, when the volume data is stored in the storage device 2, the morphological image control unit 15 reads the volume data from the storage device 2, and outputs the volume data to the image data fusing unit 17.

The functional image analyzing unit 16 extracts the active region from the functional image data, serving as the volume data, output from the functional image control unit 14 on the basis of a threshold of the physical quantity. That is, the functional image analyzing unit 16 extracts the active region to be targeted from the functional image data, serving as the volume data. Note that an active level or voxel value corresponds to the threshold of the physical quantity, the threshold of the physical quantity is predetermined in accordance with the designation of a doctor or an operator. The functional image analyzing unit 16 extracts the active region having a predetermined active level or a value equal to or more than a predetermined voxel value.

The functional image analyzing unit 16 outputs the functional image data, serving as the volume data, indicating the active region extracted by the functional image analyzing unit 16 to the image data fusing unit 17 and the image creating unit 18.

According to a well-known method, the image data fusing unit 17 fuses the functional image data, serving as the volume data, output from the functional image control unit 14 and the morphological image data, serving as the volume data, output from the morphological image control unit 15 to create first fused-image data, serving as the volume data. Herein, the image data fusing unit 17 matches a coordinate system of the functional image data, serving as the volume data, to a coordinate system of the morphological image data, serving as the volume data, and performs positioning operation. Further, the image data fusing unit 17 matches the coordinate system of the functional image data, serving as the volume data, to the voxel size of the morphological image data, serving as the volume data, thereby creating the first fused-image data, serving as the volume data (registration). Thus, it is possible to display the image obtained by fusing the morphological image and the functional image on the same space. For example, the image data fusing unit 17 fuses CT image data and PET image data expressed on the real space, to perform the positioning operation by matching the coordinate system of the CT image data and to that of the PET image data. The image data fusing unit 17 outputs the first fused-image data, serving as the volume data, to the image creating unit 18.

The description has been given of the case of creating the first fused-image data, serving as the volume data, by the image data fusing unit 17. Further, according to the similar method, the image data fusing unit 17 fuses the functional image data, serving as the volume data, indicating the active region output from the functional image analyzing unit 16 and the morphological image data, serving as the volume data, output from the morphological image control unit 15, to create second fused-image data, serving as the volume data.

The image creating unit 18 creates three-dimensional image data on the basis of the first fused-image data and the second fused-image data, serving as the volume data, output from the image data fusing unit 17. Note that the image creating unit 18 can create the three-dimensional image data on the basis of the functional image data, serving as the volume data, output from the functional image control unit 14 and the morphological image data, serving as the volume data, output from the morphological image control unit 15. The image creating unit 18 executes a three-dimensional display method, such as volume rendering or surface rendering, of the volume data, thereby creating three-dimensional image data for observing the active region and three-dimensional image data indicating the appearance of a diagnostic portion.

Specifically, the image creating unit 18 comprises a parallel-projection image creating section 18a and a perspective-projection image creating section 18b. The parallel-projection image creating section 18a creates three-dimensional image data for display operation on the basis of the volume data with so-called parallel projection. On the other hand, the perspective-projection image creating section 18b creates three-dimensional image data for display operation on the basis of the volume data with so-called perspective projection. Note that the three-dimensional image data indicates that image data is created on the basis of the volume data and is displayed on a monitor of the display device 4.

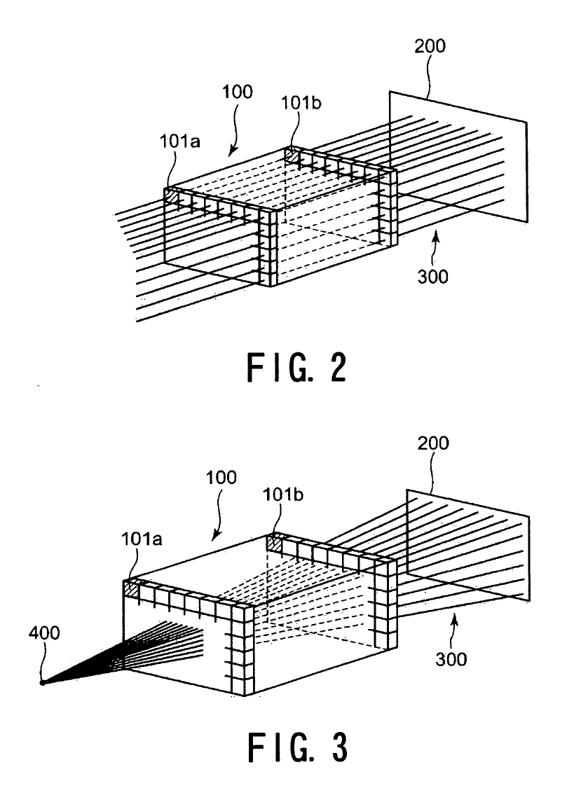

Herein, a description is given of the volume rendering that is executed by the parallel-projection image creating section 18a and the perspective-projection image creating section 18b with reference to FIGS. 2 and 3. FIG. 2 is a drawing for explaining the parallel projection, that is, processing for creating the three-dimensional image data with the parallel projection. FIG. 3 is a drawing for explaining the perspective projection, that is, processing for creating the three-dimensional image data with the perspective projection.

First, a description is given of the parallel projection executed by the parallel-projection image creating section 18a. Referring to FIG. 2, a voxel denotes minute unit regions (101a and 101b), serving as component units of a three-dimensional region (volume) of an object 100, and a voxel value denotes data specific to a characteristic of the voxel. The entire subject 100 is expressed as a three-dimensional data alignment of the voxel value, referred to as the volume data. The volume data is obtained by laminating two-dimensional tomographic image data that is sequentially obtained along the direction vertical to the tomographic surface of a targeted object. In the case of collecting the tomographic image data by the X-ray CT apparatus, the volume data is obtained by laminating the tomographic images aligned in the body axial direction at a predetermined interval. The voxel value of the voxel indicates the amount of absorption of radiation rays at the sharing position of the voxel.

The volume rendering creates the three-dimensional image on the projection surface by so-called ray casting with the above-mentioned volume data. Referring to FIG. 2, according to the ray casting, a virtual projection surface 200 is arranged on the three-dimensional space, virtual beams, referred to as rays 300, are emitted from the projection surface 200, and an image of virtual reflected light from an object (volume data) 100 is created, thereby creating a perspective image of the three-dimensional structure of the object (volume data) 100 to the projection surface 200. Specifically, light is uniformly emitted from the projection surface 200, and such a simulation of virtual physical phenomena is performed that the emitted light is reflected, attenuated, and absorbed by the object (volume data) 100 expressed by the voxel value.

With the volume rendering, the object structure can be drawn from the volume data. In particular, even when the object 100 is the human body having the complicated tissue, such as the bone or the organ, the object 100 can be drawn with separation thereof by varying and controlling the transmittance (controlling the (opacity)). That is, for a perspective portion, the opacity of the voxel forming the portion is increased and, on the other hand, for a non-perspective portion, the opacity is reduced, thereby observing the desired portion. For example, the opacity of the epidermis is reduced, thereby observing a perspective image of the blood vessel and the bone.

In the ray casting of the volume rendering, all rays 300 extended from the projection surface 200 are vertical to the projection surface 200. That is, all the rays 300 are in parallel with each other and, that is, this indicates that an observer views the object 100 from an infinite position. The method is referred to as the parallel projection and is executed by the parallel-projection image creating section 18a. Note that an operator can change the direction (hereinafter, also referred to as a line-of-sight direction) of the ray 300, relative to the volume data, in an arbitrary direction.

Next, a description is given of the perspective projection executed by the perspective-projection image creating section 18b. With the perspective projection, such it is possible to create a three-dimensional image like an image via virtual endoscopy, that is, observed from the tubular tissue, such as the blood vessel, the intestine, and the bronchi. With the perspective projection executed by the perspective-projection image creating section 18b, referring to FIG. 3, on the projection surface 200, a virtual point-of-view 400 is assumed to the opposite side of the object (volume data) 100 and all the rays 300 are radially extended via the point-of-view 400. Thus, the point-of-view 400 can be placed in the object 100 and the image that is viewed from the inside of the object 100 can be created on the projection surface 200.

With the perspective projection, the morphological image similar to that obtained by the image endoscope examination can be observed, thereby easing the pain of a patient in the examination. Further, the perspective projection can be applied to a portion or the organ, to which an endoscope cannot be inserted. Further, it is possible to obtain an image viewed from an unobservable direction with an actual endoscope, by properly changing the position of the point-of-view 400 or the line-of-sight direction (direction of the ray 300) relative to the volume data.

The image creating unit 18 outputs the three-dimensional image data to the display control unit 19.

The display control unit 19 simultaneously displays a plurality of pieces of the three-dimensional image data output from the image creating unit 18, as a plurality of three-dimensional image, on the display device 4. Further, the display control unit 19 allows the display device 4 to sequentially display a plurality of pieces of the three-dimensional image data, serving as a plurality of three-dimensional images, output from the image creating unit 18. Moreover, the display control unit 19 sequentially updates the three-dimensional image data output from the image creating unit 18 in accordance with a display updating command input from the input device 5, and allows the display device 4 to display the updated three-dimensional image data, serving as the three-dimensional image.

The display device 4 comprises a cathode ray tube (CRT) or a liquid crystal display, and displays the three-dimensional image data, serving as the three-dimensional image, under the control of the display control unit 19.

The input device 5 comprises a mouse and a keyboard. The image processing system 3 receives the position of the point-of-view 400 and the line-of-sight direction in the volume rendering, the display updating command, and a parameter, such as the opacity, with the input device 5 by an operator. The operator inputs the position of the point-of-view 400, the line-of-sight direction, or the parameter, such as the opacity, with the input device 5 and the information on the parameter is sent to the image creating unit 18. The image creating unit 18 executes the image rendering on the basis of the information on the parameter.

(Operation)

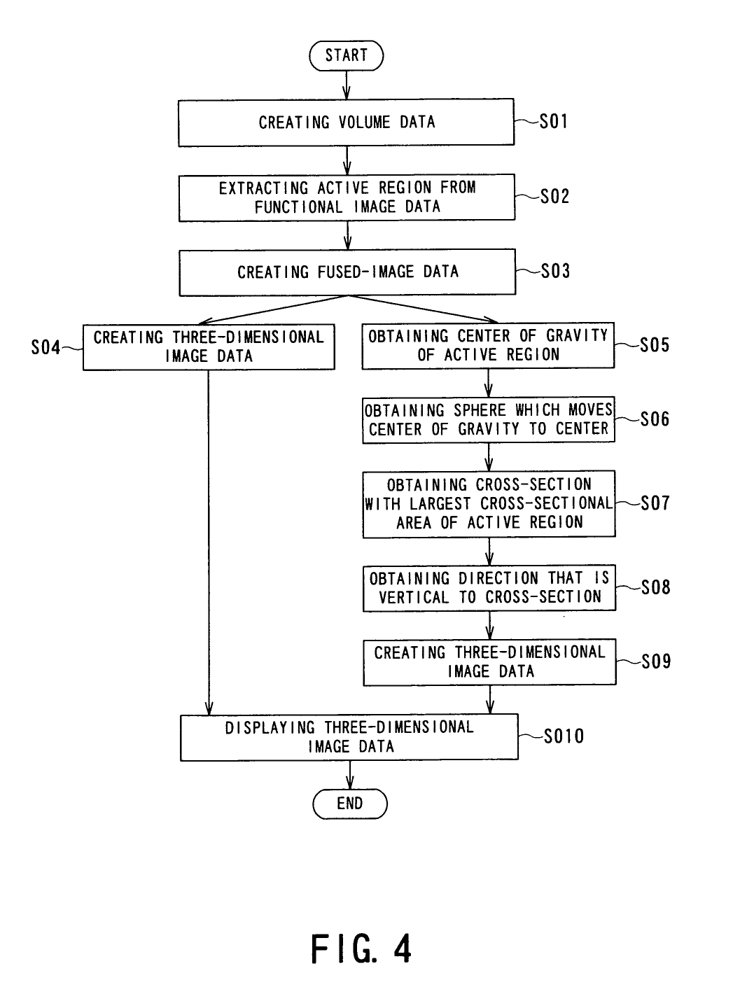

Next, a description is given of operation of the diagnostic imaging system 1 and the image processing system 3 with reference to FIGS. 1 to 12. FIG. 4 is a flowchart for an operation of the diagnostic imaging system 1 and the image processing system 3 according to the first embodiment of the present invention.

First, the functional image control unit 14 of the image processing system 3 reads a plurality of pieces of the functional image data, serving as two-dimensional image data, from the storage device 2, and creates the functional image data, serving as the volume data, expressed on the three-dimensional real space. The morphological image control unit 15 reads a plurality of pieces of the morphological image data, serving as two-dimensional image data, from the storage device 2, and creates the morphological image data, serving as the volume data, expressed on the three-dimensional real space (in step S01). Note that, when the storage device 2 stores the volume data, the functional image control unit 14 and the morphological image control unit 15 read the volume data from the storage device 2.

Subsequently, the functional image control unit 14 outputs the functional image data, serving as the volume data, to the functional image analyzing unit 16 and the image data fusing unit 17. Note that the functional image control unit 14 can output the functional image data, serving as the volume data, to the image creating unit 18.

The morphological image control unit 15 outputs the morphological image data, serving as the volume data, to the image data fusing unit 17. Note that the morphological image control unit 15 can output the morphological image data, serving as the volume data, to the image creating unit 18.

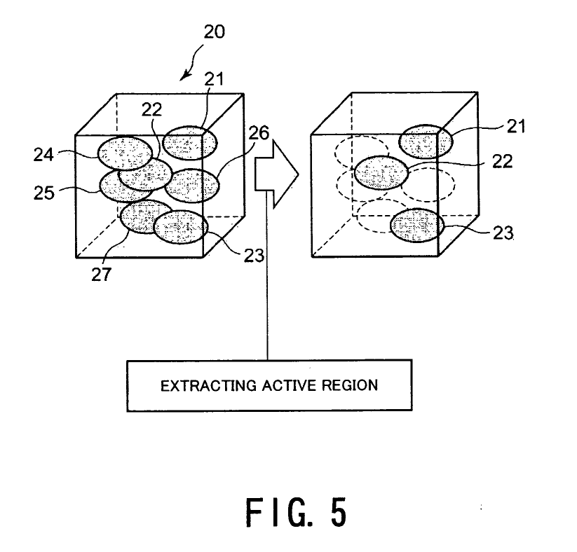

The functional image analyzing unit 16 extracts the active region from the functional image data output from the functional image control unit 14 on the basis of a predetermined threshold of the physical quantity (in step S02). As a consequence of the processing in step S02, the targeted active region is extracted from the functional image data created in the processing in step S01. The extracting processing is described with reference to FIG. 5.

FIG. 5 is a drawing for explaining the extracting processing of the active region from the functional image data, serving as the volume data.

Referring to FIG. 5, the functional image control unit 14 creates the functional image data 20, serving as the volume data, expressed on the three-dimensional real space. The functional image data 20 comprises a plurality of regions, e.g., seven regions 21 to 27. The functional image analyzing unit 16 extracts the active region from the functional image data 20 on the basis of a predetermined threshold of the physical quantity. For example, the operator's designation predetermines, as a threshold, one active level or one voxel value, and the functional image analyzing unit 16 extracts the active region having the predetermined active level or voxel value or more. Note that, in the example shown in FIG. 5, the three regions 21, 22 and 23 are the active regions.

The functional image analyzing unit 16 outputs the functional image data, serving as the volume data, indicating the active region extracted by the processing step S02 to the image data fusing unit 17 and the image creating unit 18.

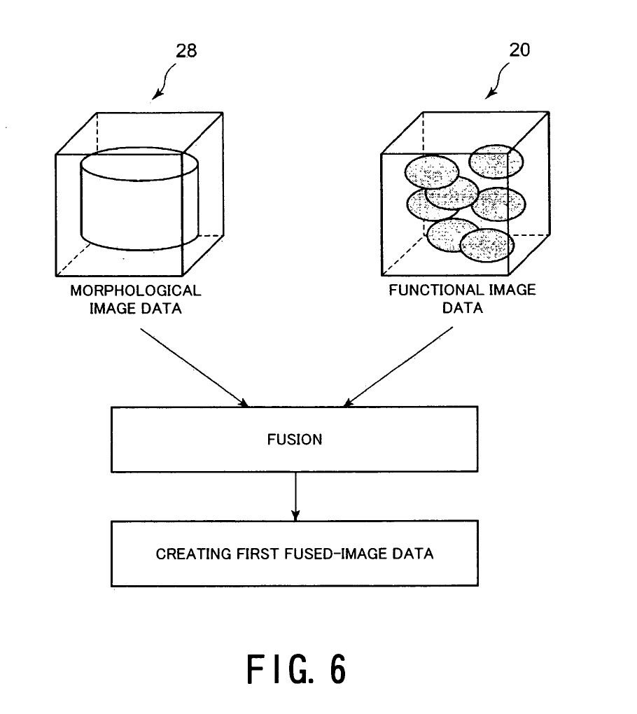

Further, the image data fusing unit 17 fuses the functional image data, serving as the volume data, output from the functional image control unit 14 and the morphological image data, serving as the volume data, output from the morphological image control unit 15, to create the first fuses-image data, serving as the volume data. Further, the image data fusing unit 17 fuses the functional image data, serving as the volume data, indicating the active region output from the functional image analyzing unit 16 and the morphological image data, serving as the volume data, output from the morphological image control unit 15, to create the second fused-image data, serving as the volume data (in step S03). The fusing processing in step S03 is described with reference to FIG. 6.

FIG. 6 is a drawing for explaining the fusing processing of the morphological image data and the functional image data. Note that FIG. 6 shows an example of the fusing processing in which the first fused-image data is created as the volume data.

Referring to FIG. 6, the image data fusing unit 17 performs positioning processing by matching a coordinate system of the functional image data 20, serving as the volume data, output from the functional image control unit 14 to a coordinate system of the morphological image data 28, serving as the volume data, output from the morphological image control unit 15. Further, the image data fusing unit 17 matches the voxel size of the functional image data 20, serving as the volume data, to the voxel size of the morphological image data 28, serving as the volume data, thereby creating the first fused-image data, serving as the volume data. Thus, the first fused-image data, serving as the volume data, expressed on the same space, is created. The first fused-image data, serving as the volume data, is output from the image data fusing unit 17 to the image creating unit 18.

Note that, in the one example, the image data fusing unit 17 creates the first fused-image data, serving as the volume data. According to the same method, the image data fusing unit 17 fuses the functional image data, serving as the volume data, indicating the active region output from the functional image analyzing unit 16 and the morphological image data, serving as the volume data, output from the morphological image control unit 15 to create the second fused-image data, serving as the volume data.

The image creating unit 18 creates the three-dimensional image data on the basis of the first fused-image data and the second fused-image data, serving as the volume data, created by the processing in step S03. The image creating unit 18 can create the three-dimensional image data on the basis of the functional image data, serving as the volume data, output from the functional image control unit 14 and the morphological image data, serving as the volume data, output from the morphological image control unit 15. The image creating unit 18 executes the three-dimensional display method, including the volume rendering and the surface rendering, of the volume data, thereby creating the three-dimensional image data (in step S04).

The processing in steps S01 to S04 creates the three-dimensional image data (superimposed image data) that is obtained by superimposing the morphological image data collected by the X-ray CT apparatus and the functional image data collected by a nuclear medical diagnosing apparatus. Note that an operator can select the parallel projection or the perspective projection with the input device 5 and the image creating unit 18 executes the volume rendering with the selected projection.

When an operator selects the parallel projection with the input device 5, the parallel-projection image creating section 18a executes the volume rendering with the parallel projection, thereby creating the three-dimensional image data. When the parallel-projection image creating section 18a creates the three-dimensional image data, an operator designates the line-of-sight direction with the input device 5 and the parallel-projection image creating section 18a thus executes the volume rendering in accordance with the designated line-of-sight direction, thereby creating the three-dimensional image data.

On the other hand, when an operator selects the perspective projection with the input device 5, the perspective-projection image creating section 18b executes the volume rendering with the perspective projection, thereby creating the three-dimensional image data. When the perspective-projection image creating section 18b creates the three-dimensional image data, an operator designates the position of the point-of-view 400 and the line-of-sight direction with the input device 5 and the perspective-projection image creating section 18b thus executes the volume rendering in accordance with the designated position of the point-of-view 400 and the designated line-of-sight direction, thereby creating the three-dimensional image data.

When the diagnostic portion includes the tubular tissue, such as the blood vessel, the intestine, or the bronchi, the perspective-projection image creating section 18b executes the volume rendering, thereby creating the three-dimensional image data via the virtual endoscopy, that is, the image data of the tubular tissue, such as the blood vessel, viewed from the inside thereof.

The display control unit 19 outputs the three-dimensional image data created by the processing in step S04 to the display control unit 19. The display control unit 19 allows the display device 4 to display the three-dimensional image data, as the three-dimensional image (in step S10).

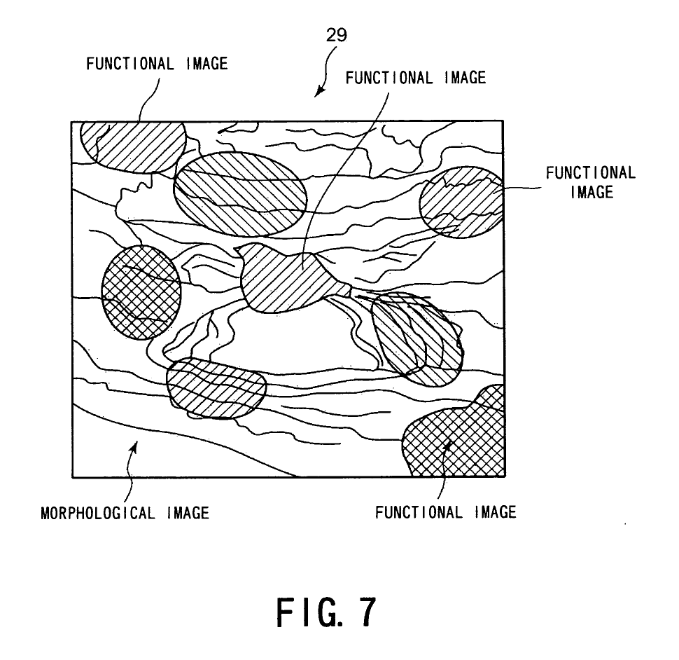

FIG. 7 is a drawing showing one example of the three-dimensional image obtained from the three-dimensional image data via the virtual endoscopy.

Referring to FIG. 7, a three-dimensional image 29 is shown. The three-dimensional image 29 is created when the perspective-projection image creating section 18b in the image creating unit 18 executes the volume rendering of the second fused-image data, serving as the volume data, output from the image data fusing unit 17. Note that, with the functional image on the three-dimensional image 29, the active region can be color-mapped with the grayscale varied depending on the activity of the active region.

When the image creating unit 18 executes the volume rendering of the first fused-image data and the second fused-image data, serving as the volume data, in the processing in step S04, an image creating condition including the opacity is input from the input device 5, and the image creating unit 18 subsequently executes the volume rendering in accordance with the image creating condition, thereby creating the three-dimensional image data. The three-dimensional image data is output to the display device 4 from the image creating unit 18 via the display control unit 19.

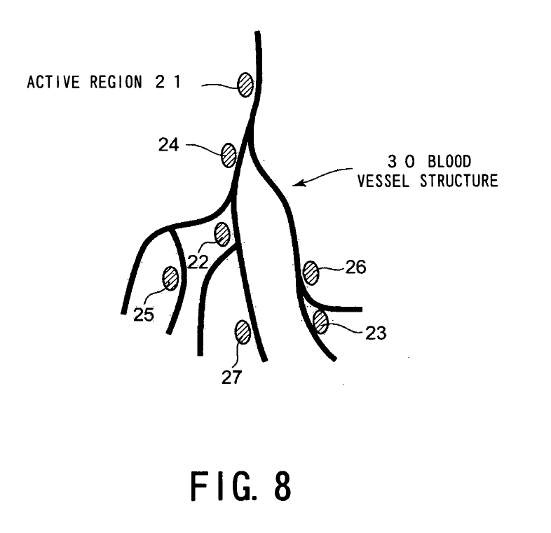

When the diagnostic portion is a tubular region, such as the blood vessel, the parallel-projection image creating section 18a or the perspective-projection image creating section 18b executes the volume rendering, thereby creating the three-dimensional image data indicating the appearance of the tubular region obtained by superimposing a blood vessel structure 30 (morphological image) and the regions 21 to 27 (functional images), serving as the active region. Herein, FIG. 8 shows one example of the three-dimensional image (blood vessel structure) 30 obtained from the three-dimensional image data indicating the appearance of the tubular region. Note that, with the functional image on the three-dimensional image 30, the active region can be color-mapped with the grayscale varied depending on the activity of the active region.

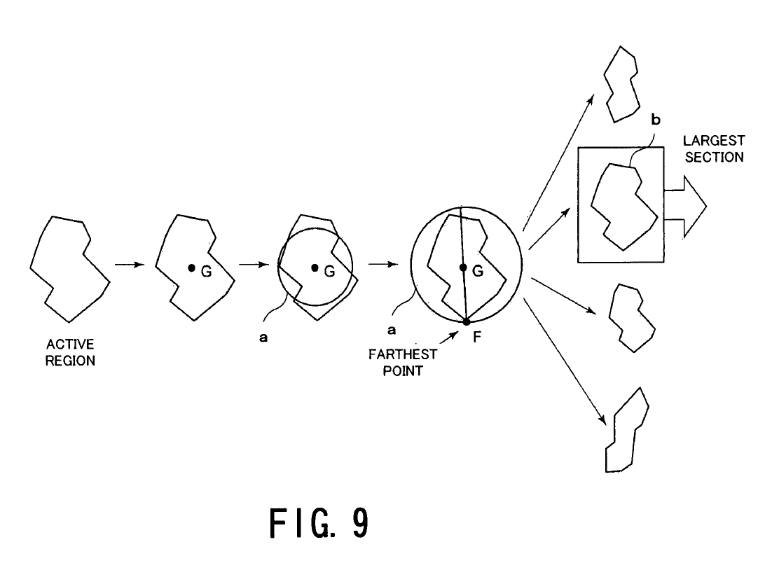

Note that the description is given of the example of determining the line-of-sight direction by the operator's designation with the input device 5. Herein, a description is given of a method for automatically determining the line-of-sight direction with reference to FIG. 9. FIG. 9 is a drawing for explaining how to determine a line-of-sight direction.

First, referring to FIG. 9, the image creating unit 18 obtains a center G of gravity of the active region existing in the functional image data, serving as the volume data, indicating the active region output from the functional image analyzing unit 16 (in step S05).

Subsequently, the image creating unit 18 obtains a sphere “a” which moves the center G of gravity obtained by the processing in step S05 to the center (in step S06), and further obtains a point F, most apart from the center of the sphere “a” in the active region, by changing the radius of the sphere “a”. Subsequently, the image creating unit 18 obtains a cross-section b with the largest cross-sectional area of the active region on the plane passing through a line segment FG connecting the farthest point F and the center G of gravity of the sphere “a” (in step S07).

Subsequently, the image creating unit 18 obtains a direction that is vertical to the cross-section b (in step S08) and, with the obtained direction as the line-of-sight direction, creates the three-dimensional image data by the volume rendering of the volume data created by the processing in step S03 (in step S09).

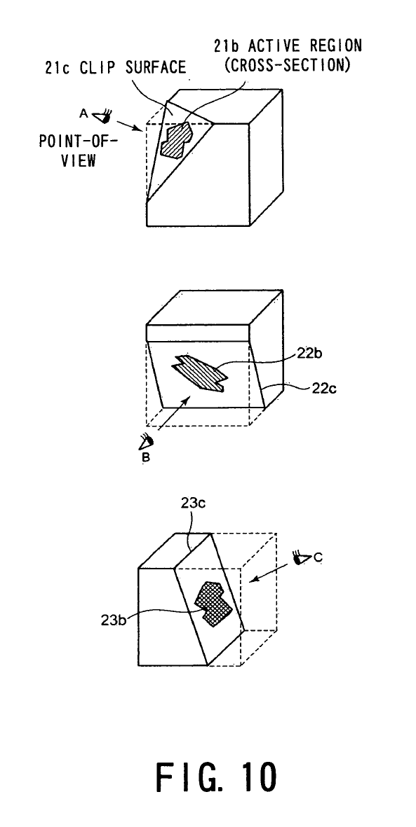

Referring to FIG. 10, a direction “A” vertical to a cross-section 21b of the region 21, serving as the active region, is set as the line-of-sight direction. Further, the three-dimensional image data is created by executing the volume rendering of the volume data created by the processing in step S03 with the parallel projection or the perspective projection. In the case of the region 22, serving as the active region, similarly, a direction B vertical to the cross-section 22b of the region 22 is set as the line-of-sight direction and the three-dimensional image data is created by executing the volume rendering of the volume data created by the processing in step S03. Further, in the case of the region 23, serving as the active region, similarly, a direction C vertical to the cross-section 23b of the region 23 is set as the line-of-sight direction and the three-dimensional image data is created by executing the volume rendering of the volume data created by the processing in step S03. When a plurality of the active regions are extracted by the processing in step S02, the image creating unit 18 creates the three-dimensional image data by automatically changing the line-of-sight direction for each of the extracted plurality of the active regions.

In the volume rendering, the image between the active region and the point-of-view out of the volume data may not be displayed by the well-known clipping processing. The clipping processing is performed by the image creating unit 18.

In the example shown in FIG. 10, the image creating unit 18 determines a clip surface 21c parallel with the cross-section 21b, further determines a clip surface 22c parallel with the cross-section 22b, and furthermore determines a clip surface 23c parallel with the cross-section 23b so as to display the cross-sections 21b, 22b, and 23b having the largest cross-sectional areas on the display device 4. The image creating unit 18 removes the volume data between the clip surfaces 21c, 22c, and 23c and the point-of-view out of the volume data, with the clip surfaces 21c, 22c, and 23c as boundaries. Thereafter, the image creating unit 18 executes the volume rendering, thereby creating the three-dimensional image data. The display control unit 19 allows the display device 4 to display the three-dimensional image data, as a three-dimensional image, created by the image creating unit 18.

That is, the display control unit 19 sets non-display operation of the three-dimensional image between the point-of-view out of the volume data and the regions 21, 22, and 23, serving as the active regions, and allows the display device 4 to display the three-dimensional image other than the above-mentioned image. Thus, it is possible to observe the active region obtained by removing the image in front of the regions 21, 22, and 23.

According to a method for determining a range for the clipping processing, the image creating unit 18 may obtain a sphere with a radius connecting the point-of-view out of the volume data and the center G of gravity of the cross-section b and may remove the image in the obtained sphere, thereby creating the three-dimensional image data. Further, the display control unit 19 allows the display device 4 to display the three-dimensional image created by the image creating unit 18. In other words, the display control unit 19 sets the non-display operation of the three-dimensional image included in the region of the obtained sphere and allows the display device 4 to display the three-dimensional image other than the image. As mentioned above, the image can be removed by automatically determining the clipping region and the active region can be displayed. Therefore, an operator can easily observe the image of the targeted active region by the operation including the clipping processing without searching the targeted active region.

The display control unit 19 outputs the three-dimensional image data created by the processing in step S09 to the display device 4, and allows the display device 4 to display the output image, as the three-dimensional image (in step S10). For example, the image creating unit 18 automatically determines the line-of-sight direction, thereby creating three types of the three-dimensional image data having the directions individually vertical to the cross-section 21b, the cross-section 22b, and the cross-section 23b, serving as the line-of-sight directions. The display control unit 19 allows the display device 4 to display the three types of the three-dimensional image data, serving as three types of three-dimensional images.

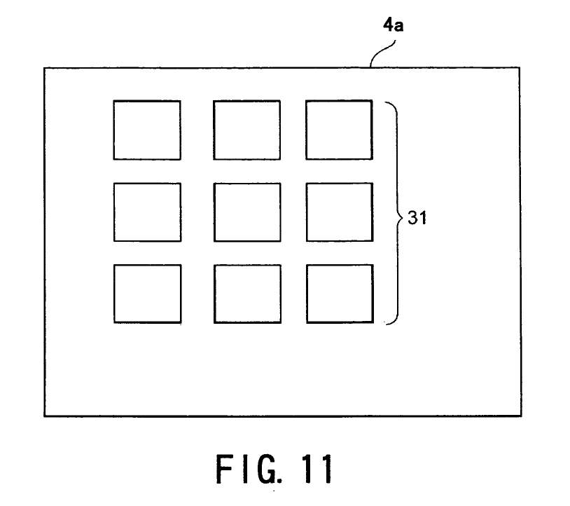

FIG. 11 is a drawing showing one example of a monitor screen of the display device 4.

Referring to FIG. 11, the display control unit 19 allows a monitor screen 4a of the display device 4 to display the three-dimensional image data for observing the active region created by the processing in step S04 or S09, as a three-dimensional image 31. Herein, the region shared by the three-dimensional image 31 is reduced on the monitor screen 4a of the display device 4 and a plurality of the three-dimensional images 31 are simultaneously displayed. That is, the display control unit 19 allows the monitor screen 4a of the display device 4 to thumbnail-display the plurality of the three-dimensional images 31. When the line-of-sight direction is automatically determined and a plurality of pieces of the three-dimensional image data are created, a plurality of three-dimensional images having different line-of-sight directions are simultaneously displayed.

Further, an arbitrary three-dimensional image 31 thumbnail-displayed on the monitor screen 4a is designated (clicked) with the input device 5, thereby enlarging and displaying the arbitrary three-dimensional image 31 on the monitor screen 4a.

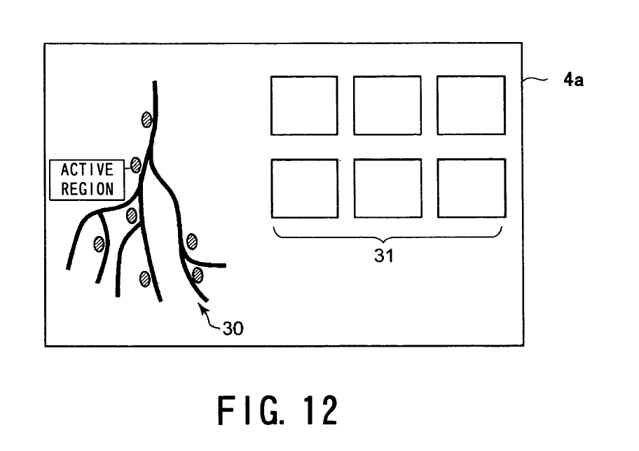

FIG. 12 is a drawing showing another example of the monitor screen of the display device 4.

Referring to FIG. 12, the display control unit 19 allows the monitor screen 4a of the display device 4 to simultaneously display a three-dimensional image (morphological image) indicating the appearance of the blood vessel structure 30 shown in FIG. 8 and the plurality of the three-dimensional images 31 created by the processing in step S04 or S09.

Note that the display format is not limited to those shown in FIGS. 11 and 12. For example, under the control of the display control unit 19, the monitor screen 4a of the display device 4 may display only the three-dimensional image data in one line-of-sight direction, created by the processing in step S04 or S09. Alternatively, when an operator selects the three-dimensional image from the plurality of the three-dimensional images 31 displayed on the monitor screen 4a, the display control unit 19 may enlarge the selected three-dimensional image and may display the enlarged image on the display device 4 by inputting information indicating the selection to the display control unit 19 from the input device 5.

When the diagnostic portion is moved and the diagnostic imaging system 1 collects the functional image data or the morphological image data on time series, the image creating unit 18 may execute the volume rendering by fixing the position of the point-of-view 400 with the perspective projection, thereby creating the three-dimensional image data. Further, when the diagnostic portion is moved and the diagnostic imaging system 1 collects the functional image data or the morphological image data on time series, the distance between the point-of-view 400 and the active region may be kept by moving the point-of-view 400 in accordance with the changing of the image data. Specifically, the volume rendering may be executed by fixing the absolute position of the point-of-view 400 on the coordinate system of the volume data. Alternatively, the volume rendering may be executed by fixing the relative positions between the point-of-view 400 and the active region. When the absolute position of the point-of-view 400 is fixed on the coordinate system, the movement of the diagnostic portion changes the distance between the point-of-view 400 and the active region, thereby executing the volume rendering in the state. On the other hand, when the point-of-view 400 is moved in accordance with the movement of the diagnostic portion to fix the relative positions between the point-of-view 400 and the active region, a constant distance between the point-of-view 400 and the active region is kept, thereby executing the volume rendering in the state. That is, the image creating unit 18 changes the position of the point-of-view 400 in accordance with the movement of diagnostic portion so as keep the constant distance between the point-of-view 400 and the active region, and creates the three-dimensional image data by executing the volume rendering at each position.

With the diagnostic imaging system 1 and the image processing system 3 according to the present invention, the active region is extracted from the functional image data on the basis of the threshold of the physical quantity, the display device 4 simultaneously displays a plurality of superimposed images created by varying the line-of-sight direction depending on the active region, thereby deleting the search time of the image indicating the targeted active region. Thus, it is possible to efficiently make a diagnosis and a diagnostic reading by a doctor or the like. Further, the display device 4 simultaneously displays a plurality of superimposed images indicating the targeted active region, thereby sufficiently the diagnostic information to a doctor or the like.

### Second Embodiment

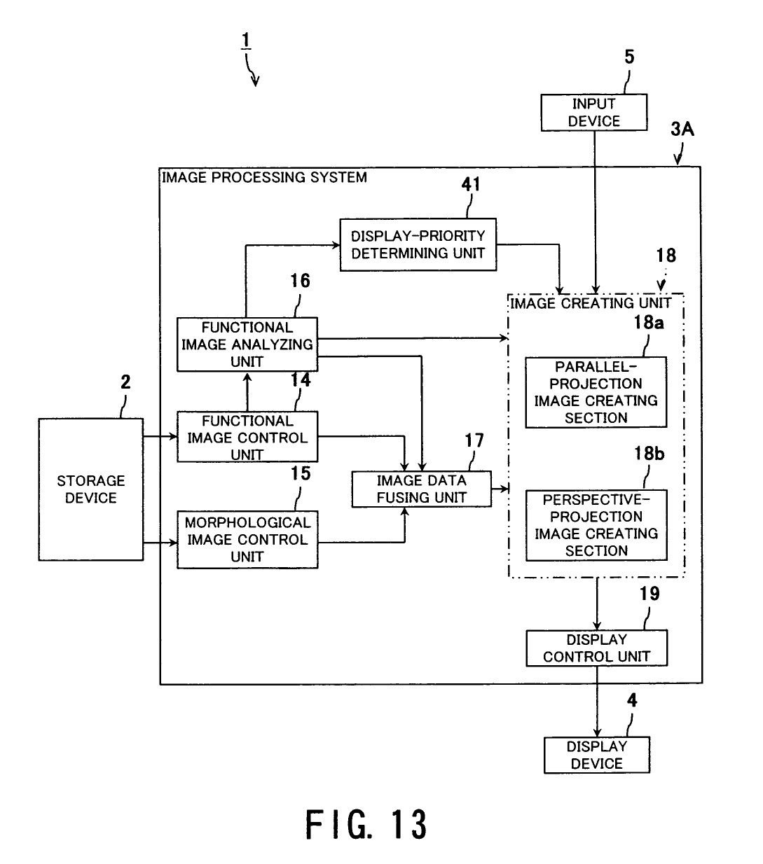

FIG. 13 is a block diagram showing a structure of a diagnostic imaging system and an image processing system according to a second embodiment of the present invention.

Referring to FIG. 13, a diagnostic imaging system 1A is shown and the diagnostic imaging system 1A comprises the storage device 2, an image processing system 3A, the display device 4 and the input device 5. Note that the diagnostic imaging system 1A includes therein the storage device 2, the image processing system 3A, the display device 4, and the input device 5, as shown in FIG. 13. However, the present invention is not limited to this structure. For example, diagnostic imaging system 1A may externally have a part or all of the storage device 2, the image processing system 3A, the display device 4, and the input device 5.

The image processing system 3A comprises the units 14 to 19 arranged to the image processing system 3 described with reference to FIG. 1 and further comprises a display-priority determining unit 41. Note that the display-priority determining unit 41 may be arranged to the image processing system 3A, as hardware, and, alternatively, may function as software. Further, referring to FIG. 13, the same reference numerals as those shown in FIG. 1 denote the same components and a description thereof is omitted.

The display-priority determining unit 41 determines the display-priority of the three-dimensional image data for observing the active region output from the functional image analyzing unit 16 on the basis of a priority determining parameter. The priority determining parameter corresponds to the volume or the active level of the active region, or the voxel value, and is selected in advance by an operator.

For example, when an operator selects, in advance, the volume of the active region, as the priority determining parameter, the display-priority determining unit 41 determines the display-priority of the three-dimensional image data for observing the active region on the basis of the volume. In this case, the display-priority determining unit 41 calculates the volume of the active region on the basis of the functional image data, serving as the volume data indicating the active region, and increases the display-priority of the active region, as the volume of the active region is larger. That is, among the active regions, the display-priority of the active region having a larger volume is increased. As mentioned above, the display-priority of the active region is determined depending on the volume of the active region and the three-dimensional image of the targeted active region thus can preferentially be displayed.

The display-priority determining unit 41 outputs, to the image creating unit 18, information indicating the display-priority of the three-dimensional image data for observing the active region.

The image creating unit 18 sequentially creates the three-dimensional image data for observing the active region in accordance with the display-priority output from the display-priority determining unit 41 on the basis of the first fused-image data and the second fused-image data, serving as the volume data, output from the image data fusing unit 17. The three-dimensional image data is sequentially output from the image creating unit 18 to the display control unit 19 in accordance with the display-priority.

The display control unit 19 allows the display device 4 to sequentially display the three-dimensional image data, as the three-dimensional image, output from the image creating unit 18, in accordance with the display-priority.

(Operation)

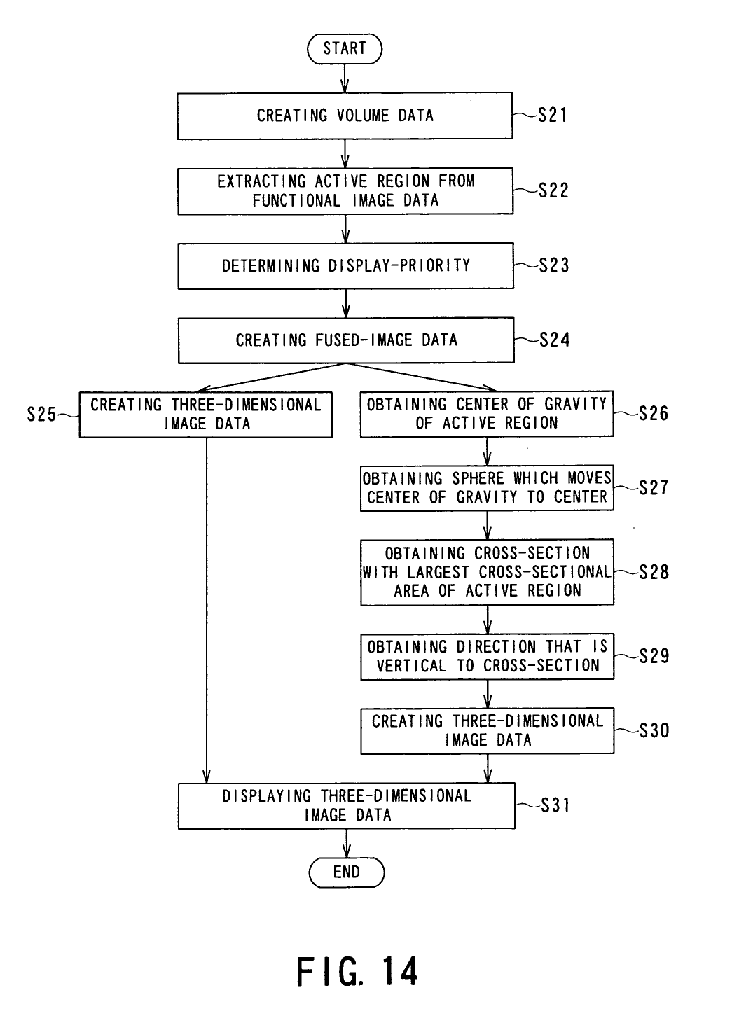

Next, a description is given of the operation of the diagnostic imaging system 1A and the image processing system 3A with reference to FIGS. 13 to 17. FIG. 14 is a flowchart for explaining an operation of the diagnostic imaging system 1A and the image processing system 3A according to the second embodiment of the present invention.

Similarly to the processing in step S01, the functional image control unit 14 in the image processing system 3A creates the functional image data, serving as the volume data. The morphological image control unit 15 creates the morphological image data, serving as the volume data (in step S21).

The functional image control unit 14 outputs the functional image data, serving as the volume data, to the functional image analyzing unit 16 and the image data fusing unit 17. The morphological image control unit 15 outputs the morphological image data, serving as the volume data, to the image data fusing unit 17.

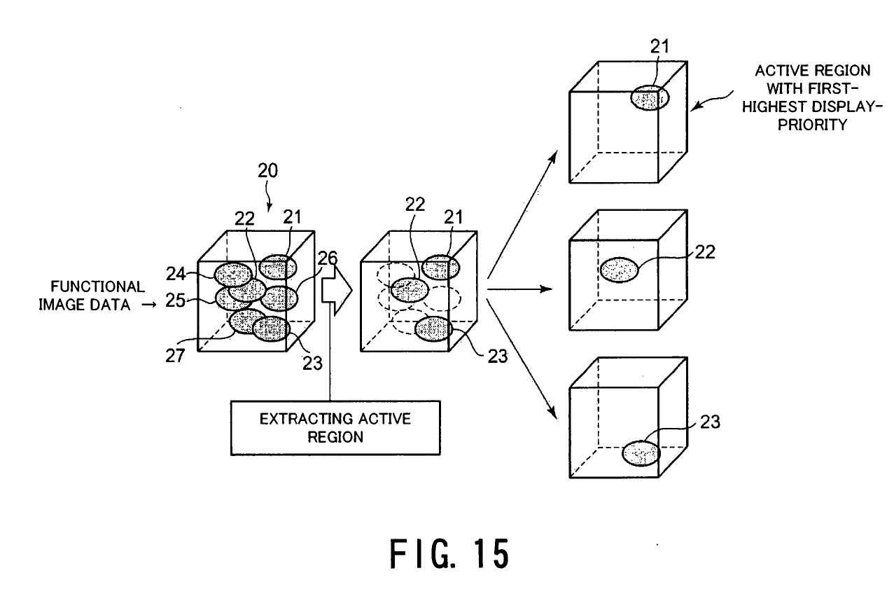

The functional image analyzing unit 16 extracts the active region from the functional image data, serving as the volume data, output from the functional image control unit 14 on the basis of a predetermined threshold of the physical quantity, similarly to the processing in step S02 (in step S22). The functional image analyzing unit 16 extracts the active region having a predetermined active level or more, or having a predetermined voxel value or more. Thus, the targeted active region is extracted. Among the active regions 21 to 27 in the example shown in FIG. 15, three regions 21, 22, and 23 are set as the active regions. The functional image data, serving as the volume data, indicating the active region is output from the functional image analyzing unit 16 to the image data fusing unit 17, the image creating unit 18, and the display-priority determining unit 41.

The display-priority determining unit 41 determines the display-priority of the three-dimensional image data for observing the active region output from the functional image analyzing unit 16 on the basis of the pre-selected priority determining parameter (in step S23). The priority determining parameter corresponds to the volume, the voxel value, or the active level of the extracted active region, and is selected in advance by an operator.

For example, upon determining the display-priority of the three-dimensional image data on the basis of the volume of the active region, as the active region has a larger volume, the display-priority determining unit 41 increases the display-priority on the basis of the functional image data, serving as the volume data, indicating the active region. Further, the display-priority determining unit 41 increases the display-priority of the active region having a larger volume so as to sequentially display the active region in order of a larger volume. For example, when the volume of the region 21 is the largest among the regions 21, 22, and 23, serving as the active regions, shown in FIG. 15, the three-dimensional image data for observing the region 21 is determined with the first-highest display-priority.

Further, the display-priority determining unit 41 determines the display-priority of the three-dimensional image for observing the region 22, serving as the active region. Moreover, the display-priority determining unit 41 determines the display-priority of the three-dimensional image data for observing the region 23, serving as the active region. In addition, the display-priority determining unit 41 determines the display-priority of the three-dimensional image data for the plurality of the active regions. Information indicating the display-priority is output to the image creating unit 18 from the display-priority determining unit 41.

Upon determining the display-priority on the basis of the voxel value or the active level, the display-priority determining unit 41 increases the display-priority in order of a larger voxel value of the active region, or increases the display-priority in order of a larger active level of the active region, thereby determining the display-priority of the three-dimensional image data for the plurality of the active region.

As mentioned above, the display-priority of the three-dimensional image data is determined on the basis of the volume or the active level of the active region, thereby preferentially displaying the three-dimensional image data for observing the targeted active region.

Similarly to the processing in step S03, the image data fusing unit 17 fuses the functional image data, serving as the volume data, and the morphological image data, serving as the volume data, to create the first fused-image data and the second fused-image data, serving as the volume data (in step S24). The first fused-image data and the second fused-image data is output to the image creating unit 18 from the image data fusing unit 17.

The image creating unit 18 creates the three-dimensional image data on the basis of the first fused-image data and the second fused-image data, serving as the volume data, output from the image data fusing unit 17. The image creating unit 18 creates the three-dimensional image data by executing the volume rendering of the volume data (in step S25).

In step S25, the image creating unit 18 sequentially creates the three-dimensional image data in accordance with the display-priority of the three-dimensional image data determined by the processing in step S23, and sequentially outputs the three-dimensional image data to the display control unit 19. When the first display-priority is determined to the three-dimensional image data for observing the region 21, serving as the active region, the second display-priority is determined to the three-dimensional image data for observing the region 22, serving as the active region, and the third display-priority is determined to the three-dimensional image data for observing the region 23, serving as the active region, the image creating unit 18 sequentially creates three types of the three-dimensional image data for observing the regions 21, 22, and 23 in accordance with the display-priority and outputs the created image data to the display control unit 19.

The display control unit 19 allows the display device 4 to sequentially display the three-dimensional image data, serving as the three-dimensional image, for observing the active region, in accordance with the display-priority (in step S31). Note that the image creating unit 18 may create only the three-dimensional image data for observing the active region with the highest display-priority among a plurality of the active regions. In this case, the display control unit 19 allows the display device 4 to display only the three-dimensional image data for observing the active region with the highest display-priority.

Note that the operator designates the position of the point-of-view and the line-of-sight direction with the input device 5 upon executing the volume rendering. Alternatively, the line-of-sight direction is automatically determined by setting the direction vertical to the cross-section having the largest cross-sectional area of the active region, as described in steps S05 to S08 with reference to FIGS. 9 and 10. Note that, similarly to the first embodiment, an operator selects the parallel projection or the perspective projection, thereby executing the volume rendering.

Upon automatically determining the line-of-sight direction, referring to FIG. 9, the image creating unit 18 obtains the center G of gravity of the active region existing in the functional image data, serving as the volume data, indicating the active region extracted by the processing in step S22 (in step S26).

Subsequently, the image creating unit 18 obtains the sphere “a” which moves the center G of the gravity obtained by the processing in step S26 to the center (in step S27). Further, the image creating unit 18 obtains the point F, most apart from the center of the sphere “a” in the active region by changing the radius of the sphere “a”. Furthermore, the image creating unit 18 obtains the cross-section b with the largest cross-sectional area of the active region on the plane passing through the line segment FG connecting the farthest point F and the center G of gravity of the sphere “a” (in step S28).

Subsequently, the image creating unit 18 obtains a direction that is vertical to the cross-section b (in step S29). The image creating unit 18 creates the three-dimensional image data by executing the volume rendering of the volume data in the obtained direction, as the line-of-sight direction (in step S30).

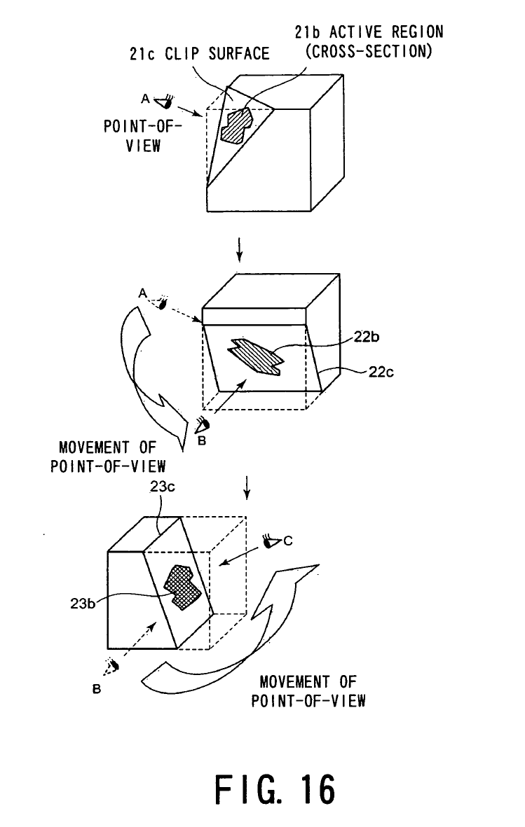

Referring to FIG. 16, the image creating unit 18 executes the volume rendering by varying the line-of-sight direction depending on the active region. The image creating unit 18 executes the volume rendering of the volume data created by the processing in step S24 in the direction A vertical to the cross-section 21b of the region 21, serving as the active region, corresponding to the line-of-sight direction, thereby creating the three-dimensional image data for observing the area 21.

Similarly in the case of the region 22, serving as the active region, the image creating unit 18 executes the volume rendering of the volume data created by the processing in step S24 in the direction B vertical to the cross-section 22b of the region 22, corresponding to the line-of-sight direction, thereby creating the three-dimensional image data for observing the region 22. Further, similarly in the case of the region 23, serving as the active region, the image creating unit 18 executes the volume rendering of the volume data created by the processing in step S24 with the direction C vertical to the cross-section 23b of the region 23, corresponding to the line-of-sight direction, thereby creating the three-dimensional image data for observing the region 23. Thus, the image creating unit 18 sequentially creates a plurality of pieces of the three-dimensional image data in the directions automatically obtained from a plurality of the active regions, corresponding to the line-of-sight directions.

Similarly to the first embodiment, the image between the point-of-view and the active region may not be displayed by the well-known clipping processing. Referring to FIG. 16, clip surfaces 21c, 22c, and 23c are determined and the image is removed with the obtained clip surfaces 21c, 22c, and 23c, as borders, and the active regions thus can be observed.

Subsequently, the display control unit 19 sequentially outputs the three-dimensional image data to the display device 4 in accordance with the display-priority determined by the processing in step S23, and allows the display device 4 to sequentially display the three-dimensional image data, as a three-dimensional image (in step S31).

For example, the display-priority determining unit 41 determines the first display-priority to the three-dimensional image data for observing the region 21, serving as the active region, further determines the second display-priority to the three-dimensional image data for observing the region 22, serving as the active region, and furthermore determines the third display-priority to the three-dimensional image data for observing the region 23, serving as the active region. In this case, the display control unit 19 first allows the display device 4 to display the three-dimensional image data created in the direction A corresponding to the line-of-sight direction, serving as the three-dimensional image, further allows the display device 4 to display the three-dimensional image data created in the direction B corresponding to the line-of-sight direction, serving as the three-dimensional image, and furthermore allows the display device 4 to display the three-dimensional image data created in the direction C corresponding to the line-of-sight direction, as the three-dimensional image. Thus, as shown in FIG. 16, the three-dimensional image is displayed like the movement of the point-of-view 400 from the direction A to the direction B and the movement of the point-of-view 400 from the direction B to the direction C.

First, the display control unit 19 allows the display device 4 to display the three-dimensional image data, serving as the three-dimensional image, created in the direction “A”, corresponding to the line-of-sight direction, relative to the active region with the first-highest display-priority. After that, an operator issues a command for updating the image display operation (moving command of the point-of-view) with the input device 5. In this case, the display control unit 19 may allow the display device 4 to display the three-dimensional image data, serving as the three-dimensional image, created in the direction B relative to the active region with the second-highest display-priority corresponding to the line-of-sight direction, thereby updating the image. Subsequently, the display control unit 19 receives the command (moving command of the point-of-view) for updating the image display operation and thus allows the display device 4 to display the three-dimensional image data, serving as the three-dimensional image, created in the direction C corresponding to the line-of-sight direction. As mentioned above, the display device 4 displays the three-dimensional image data in the changed direction, serving as the three-dimensional image, and the three-dimensional image is therefore displayed like the movement of the point-of-view.

Further, the image may be updated after the passage of a predetermined time without waiting for the command from the operator. In this case, the display control unit 19 has a counter that counts the time, and allows the display device 4 to display the three-dimensional image data indicating the next active-region after the passage of a predetermined time. Thus, the three-dimensional images are sequentially displayed by updating the three-dimensional images in the higher order of the display-priority.

Note that the monitor screen 4a of the display device 4 may simultaneously display a plurality of the three-dimensional images 31, as mentioned above with reference to the display examples shown in FIGS. 11 and 12 according to the first embodiment, and may display the plurality of the three-dimensional images 31 for observing the active region in addition to the three-dimensional image 30 indicating the appearance of the diagnostic portion. For example, the display control unit 19 allows the monitor 4a of the display device 4 to thumbnail-display the plurality of the three-dimensional images for observing the active region. Further, the display control unit 19 allows the display device 4 to enlarge and display the three-dimensional image with the highest display-priority among the plurality of the three-dimensional images displayed on the display device 4. Subsequently, when the display control unit 19 receives the command (moving command of the point-of-view) for updating the image display operation from the operator, or after the passage of a predetermined time, the display control unit 19 may allow the display device 4 to display the three-dimensional image with the second-highest display-priority, instead of the three-dimensional image with the first-highest display-priority.

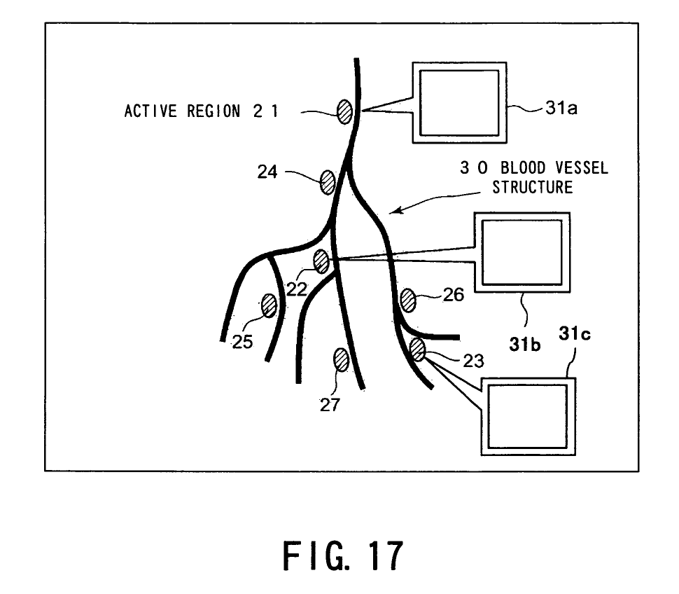

FIG. 17 is a drawing showing one example of the monitor screen of the display device 4.

Referring to FIG. 17, the display control unit 19 allows the display operation of the three-dimensional image 31 for observing the active region on the blood vessel structure 30 corresponding to the three-dimensional image indicating the appearance of the diagnostic portion. For example, the display control unit 19 allows the display operation, with a balloon, of the three-dimensional image 31 for observing the active region near the active region on the blood vessel structure 30.

Note that the blood vessel structure 30 shown in FIG. 17 is created on the basis of the first fused-image data, serving as the volume data, created by the processing in step S24. Preferably, the three-dimensional image 31 for observing the active region is created on the basis of the second fused-image data, serving as the volume data, created by the processing in step S24.

Specifically, the display control unit 19 allows the display operation, with a balloon, of a three-dimensional image 31a for observing the region 21, serving as the active region, near the region 21 of the blood vessel structure 30. Further, the display control unit 19 allows the display operation, with a balloon, of a three-dimensional image 31b for observing the region 22, serving as the active region, near the region 22 on the blood vessel structure 30. Furthermore, the display control unit 19 allows the display operation, with a balloon, of a three-dimensional image 31c for observing the region 23, serving as the active region, near the region 23 on the blood vessel structure 30.

The display screen shown in FIG. 17 clarifies the corresponding relationship between the blood vessel structure 30 and the three-dimensional images 31a, 31b, and 31c for observing the active region. Therefore, the display screen shown in FIG. 17 enables the efficient interpretation.

Upon moving the diagnostic portion and collecting the functional image data and the morphological image data on time series, similarly to the first embodiment, the image creating unit 18 keeps a constant distance between the point-of-view 400 and the active region by varying the position of the point-of-view 400 depending on the movement of the diagnostic portion, ad executes the volume rendering at each position, thereby creating the three-dimensional image data. Alternatively, the volume rendering may be executed by fixing the point-of-view 400.

With the diagnostic imaging system 1A and the image processing system 3A according to the present invention, the display-priority is determined depending on the active level or the volume of the active region, the superimposed image is created by varying the line-of-sight direction depending on the display-priority, and the created image is sequentially displayed. As a consequence thereof, the targeted active region can be preferentially displayed and observed. Thus, it is possible to efficiently make a diagnosis and a diagnostic reading by the doctor or the like, because a time for searching the targeted active region by the doctor or the like can be reduced.

### Third Embodiment

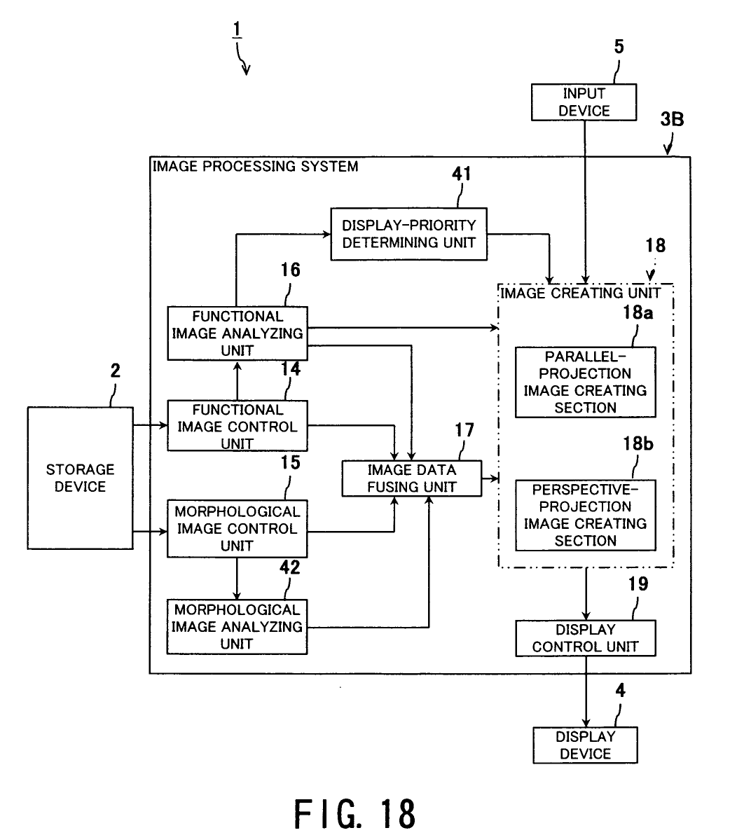

FIG. 18 is a block diagram showing a structure of a diagnostic imaging system and an image processing system according to a third embodiment of the present invention.

Referring to FIG. 18, a diagnostic imaging system 1B is shown and the diagnostic imaging system 1B comprises the storage device 2, a image processing system 3B, the display device 4, and the input device 5. Although the diagnostic imaging system 1B includes the storage device 2, the image processing system 3B, the display device 4, and the input device 5, as shown in FIG. 18, the present invention is not limited to this structure. The diagnostic imaging system 1B may externally have a part or all of the storage device 2, the image processing system 3B, the display device 4, and the input device 5.

The image processing system 3B comprises a morphological image analyzing unit 42 in addition to the units arranged to the image processing system 3A described with reference to FIG. 13. According to the third embodiment, a description is given of the case of executing the display operation via virtual endoscopy. Note that the morphological image analyzing unit 42 may be arranged to the image processing system 3B as hardware and, alternatively, may function as software. Referring to FIG. 18, the same reference numerals as those shown in FIGS. 1 and 13 denote the same components and a detailed description thereof is omitted.

The morphological image analyzing unit 42 extracts (segments) the morphological image data, serving as the volume data, indicating the tubular region (e.g., the blood vessel, the intestine, and the bronchi) from among the morphological image data, serving as the volume data. Further, the morphological image analyzing unit 42 performs thinning processing of the morphological image data, serving as the volume data, indicating the tubular region. The morphological image analyzing unit 42 outputs the morphological image data, serving as the volume data, of the tubular region subjected to the thinning processing to the image data fusing unit 17. Although not shown, the morphological image analyzing unit 42 can output the morphological image data, serving as the volume data, of the tubular region subjected to the thinning processing to the image creating unit 18.

The image data fusing unit 17 positions the functional image data, serving as the volume data, indicating the active region output from the functional image analyzing unit 16 and the morphological image data, serving as the volume data, of the tubular region output from the morphological image analyzing unit 42, fuses the functional image data and the morphological image data, to create third fused-image data, serving as the volume data.

The display-priority determining unit 41 determines the display-priority of paths on the basis of the third fused-image data, serving as the volume data, output from the image data fusing unit 17. When the tubular region is branched to a plurality of paths, the display-priority determining unit 41 determines the display-priority of the paths.

Specifically, the display-priority determining unit 41 extracts the path from among a plurality of branched tubular regions on the basis of the third fused-image data, serving as the volume data, output from the image data fusing unit 17, and obtains the relationship between the extracted path and the active region therearound. For example, the display-priority determining unit 41 obtains the distance to the active region around the extracted path, the number of the active regions around the extracted path, the voxel value of the active region around the extracted path, and the active level of the active region around the extracted path. The display-priority determining unit 41 determines the display-priority of the path whose image is displayed via virtual endoscopy on the basis of the relationship between the extracted path and the active region around the extracted path. For example, as the distance between the path and the active region around the path is shorter and the number of the active regions around the path is larger, the display-priority increases.

As mentioned above, the display-priority of the path is determined depending on the relationship between the path and the active region on the basis of the third fused-image data, serving as the volume data, and the three-dimensional image along the targeted path can be preferentially displayed.

Note that the display-priority determining unit 41 may determine the display-priority of the path on the basis of the functional image data, serving as the volume data, output from the functional image analyzing unit 16.

The display-priority determining unit 41 outputs information indicating the display-priority of the path, to the image creating unit 18.

The image creating unit 18 executes the volume rendering of the first fused-image data, the second fused-image data, and the third fused-image data, serving as the volume data, output from the image data fusing unit 17, along the path with higher display-priority, in accordance with the display-priority determined by the display-priority determining unit 41, thereby creating the three-dimensional image data. Especially, in the execution of the display operation via the virtual endoscopy, the perspective-projection image creating section 18b executes the volume rendering with the perspective projection, thereby creating the three-dimensional image via the virtual endoscopy.

(Operation)

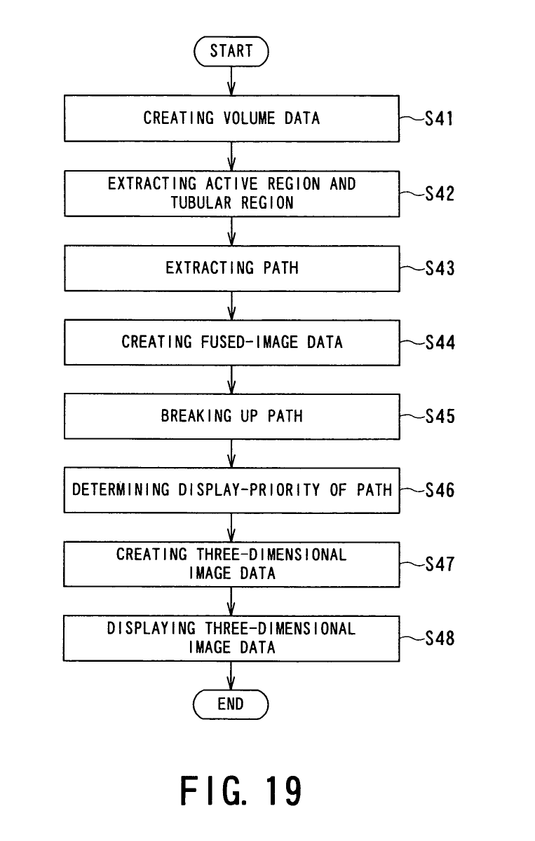

Next, a description is given of the operation of the diagnostic imaging system 1B and the image processing system 3B according to the third embodiment of the present invention with reference to FIGS. 18 to 24. FIG. 19 is a flowchart showing an operation of the diagnostic imaging system 1B and the image processing system 3B according to the third embodiment of the present invention.

First, similarly to the processing in step S01, the functional image control unit 14 in the image processing system 3B creates the functional image data, serving as the volume data, and the morphological image control unit 15 creates the morphological image data, serving as the volume data (in step S41).

The functional image control unit 14 outputs the functional image data, serving as the volume data, to the functional image analyzing unit 16 and the image data fusing unit 17. The morphological image control unit 15 outputs the morphological image data, serving as the volume data, to the image data fusing unit 17 and the morphological image analyzing unit 42.

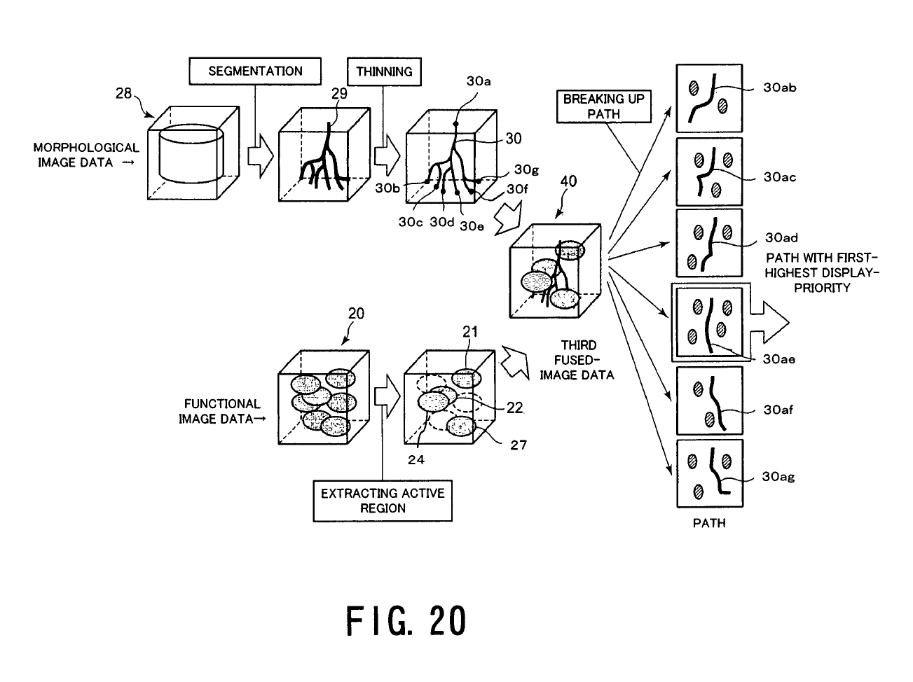

Similarly to the processing in step S02, the functional image analyzing unit 16 extracts one active region from among a plurality of the active regions existing in the functional image data 20, serving as the volume data, output from the functional image control unit 14 on the basis of a predetermined threshold of the physical quantity (in step S42). In the example shown in FIG. 20, the functional image analyzing unit 16 extracts the active regions 21, 22, 24, and 27, serving as the active region, from the functional image data 20, serving as the volume data, on the basis of the threshold of the physical quantity. The functional image data, serving as the volume data, indicating the active region is output to the image data fusing unit 17 and the image creating unit 18.

As shown in FIG. 20, the morphological image analyzing unit 42 extracts a tubular region 29 including the blood vessel, existing in the morphological image data 28, serving as the volume data (in step S42).

Further, for the purpose of simplifying the processing of the display-priority determining unit 41, the morphological image analyzing unit 42 performs thinning processing of the tubular region 29, and extracts a path 30 upon creating and displaying an image via virtual endoscopy (in step S43). The morphological image data, serving as the volume data, indicating the path 30 is output to the image data fusing unit 17 from the morphological image analyzing unit 42.

The image data fusing unit 17 fuses the functional image data, serving as the volume data, and the morphological image data, serving as the volume data, to create the first fused-image data and the second fused-image data, serving as the volume data. Further, the image data fusing unit 17 positions the functional image data, serving as the volume data, indicating the active region, output from the functional image analyzing unit 16 to the morphological image data, serving as the volume data, indicating the path 30 output from the morphological image analyzing unit 42, fuses the functional image data and the morphological image, to create the third fused-image data, serving as the volume data (in step S44). The image data fusing unit 17 outputs, to the image creating unit 18, the first fused-image data, the second fused-image data, and the third fused-image data, serving as the volume data. Further, the image data fusing unit 17 outputs the third fused-image data, serving as the volume data, to the display-priority determining unit 41.

The display-priority determining unit 41 breaks up the path 30 having a plurality of branches into a plurality of paths on the basis of the third fused-image data, serving as the volume data, output from the image data fusing unit 17 (in step S45). In the example shown in FIG. 20, the path 30 has six end points 30b to 30g relative to one start point 30a and the display-priority determining unit 41 therefore breaks up the path 30 into six paths 30ab, 30ac, 30ad, 30ae, 30af, and 30ag.

Subsequently, the display-priority determining unit 41 determines the display-priority of the path to the path on the basis of the relationship between the path and the active region existing around and the periphery thereof (in step S46). In the example shown in FIG. 20, the display-priority determining unit 41 determines the path 30ae with the highest priority for display operation on the basis of the distance between the path and the regions 21, 22, 24, and 27, serving as the active regions existing around the path, and the number of the active regions existing around the path. Further, the display-priority determining unit 41 determines the display-priority of the path for display operation, next to the path 30ae. In the example, the path is broken up into six paths and first to sixth display priorities are determined to the paths.

As mentioned above, the image along the targeted path can be preferentially displayed by determining the display-priority of the path on the basis of the relationship between the path and the active region existing around the path.

Information indicating the display-priority of the path is output from the display-priority determining unit 41 to the image creating unit 18.

In the display operation via the virtual endoscopy, the perspective-projection image creating section 18b in the image creating unit 18 executes the volume rendering with the perspective projection along the path in accordance with the display-priority determined by the processing in step S46 on the basis of the volume data output from the image data fusing unit 17, thereby creating the three-dimensional image data via the virtual endoscopy (in step S47). The three-dimensional image data is output from the image creating unit 18 to the display control unit 19.

The display control unit 19 allows the display device 4 to display the three-dimensional image data, as the three-dimensional image, created along the path in accordance with the display-priority determined by the processing in step S46 (in step S48). Thus, the display device 4 displays the three-dimensional image via the virtual endoscopy, like viewing the tubular region, such as the blood vessel, from the inside as shown in FIG. 7.

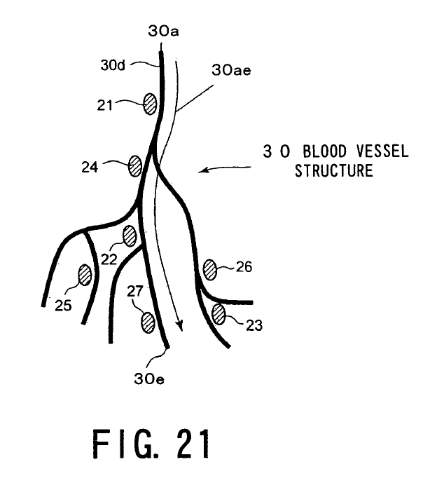

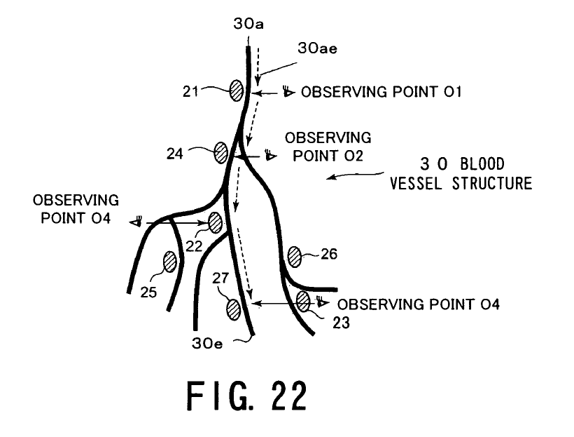

FIGS. 21 and 22 are drawings showing the path displayed via the virtual endoscopy. Since the processing in step S46 determines the path 30ae, as the highest display-priority, the perspective-projection image creating section 18b in the image creating unit 18 executes the volume rendering along the path 30ae, thereby creating the three-dimensional image data via the virtual endoscopy from the start point 30a to the end point 30e along the path 30ae. In this case, an operator determines the distance between the point-of-view 400 and the volume data, and the three-dimensional image is created on the projection surface 200 with the rays 300 radially-extended from the point-of-view 400. The perspective-projection image creating section 18b executes the volume rendering in the direction vertical to the cross-section of the path 30ae, serving as the line-of-sight direction, thereby creating the three-dimensional image data so that the point-of-view 400 exists on the inner surface of the tubular region.

As mentioned above, the display-priority of the path is determined on the basis of the third fused-image data and the three-dimensional image data along the targeted path can be preferentially created and displayed. In other words, the targeted path is automatically determined on the basis of the functional image data. Thus, it is possible to reduce the time for searching the targeted active region and the diagnosis becomes efficient. The three-dimensional image data is automatically created and displayed along the targeted path without determining the path at the branch point of the tubular region and the diagnosis thus becomes efficient.

In the case of creating the three-dimensional image data from the start point 30a to the end point 30e of the path 30ae, the perspective-projection image creating section 18b may create the three-dimensional image data every predetermined time interval and the created three-dimensional image data may be displayed, as the three-dimensional image, on the monitor screen of the display device 4. That is, the three-dimensional image data is sequentially created on the path 30ae shown in FIG. 21 every predetermined interval and it is thus possible to sequentially create and display the three-dimensional image data of the regions 21, 24, 22, and 27 corresponding to the active regions, as the three-dimensional images. The reduction of the interval causes the display operation of the three-dimensional image on the display device 4 so that the point-of-view 400 is continuously moved. In this case, the perspective-projection image creating section 18b sequentially creates the three-dimensional image data via the virtual endoscopy along the path 30ae every predetermined interval, and outputs the created three-dimensional image data to the display control unit 19. The display control unit 19 outputs the three-dimensional image data to the display device 4, and allows the display device 4 to sequentially display the three-dimensional image data, as the three-dimensional image.

Further, the three-dimensional image data may be created and displayed every active region existing along the path 30ae. In the example shown in FIG. 22, the regions 21, 24, 22, and 27, as the active regions, exist along the path 30ae. Therefore, the image creating unit 18 sequentially creates the three-dimensional image data of the regions 21, 24, 22, and 27. First, the perspective-projection image creating section 18b executes the volume rendering at an observing point O1 and the image creating unit 18 thus creates the three-dimensional image data. Subsequently, the perspective-projection image creating section 18b executes the volume rendering in the order of observing points O2, O3, and O4 and the image creating unit 18 thus sequentially creates the three-dimensional image data at the observing points O2 to O4. The three-dimensional image data is sequentially output to the display control unit 19, and the display control unit 19 allows the display device 4 to sequentially display the three-dimensional image data, serving as the three-dimensional images, in the created order.

As mentioned above, the three-dimensional image data is created every active region and the three-dimensional image data is not thus created between the active regions. For example, the three-dimensional image data is not created between the observing points O1 and O2 and the three-dimensional image data is not further created between the observing points O2 and O3 and between the observing points O3 and O4. Thus, the display device 4 displays the three-dimensional image so that the point-of-view is discretely moved.

Further, similarly to the second embodiment, when an operator issues a command for updating the image display operation (command for moving the point-of-view) with the input device 5, the display control unit 19 may allow the display device 4 to sequentially display the three-dimensional image data, serving as the three-dimensional image, created along the path in accordance with the updating command. Furthermore, the image may be automatically updated after a predetermined time without waiting for a command from an operator.

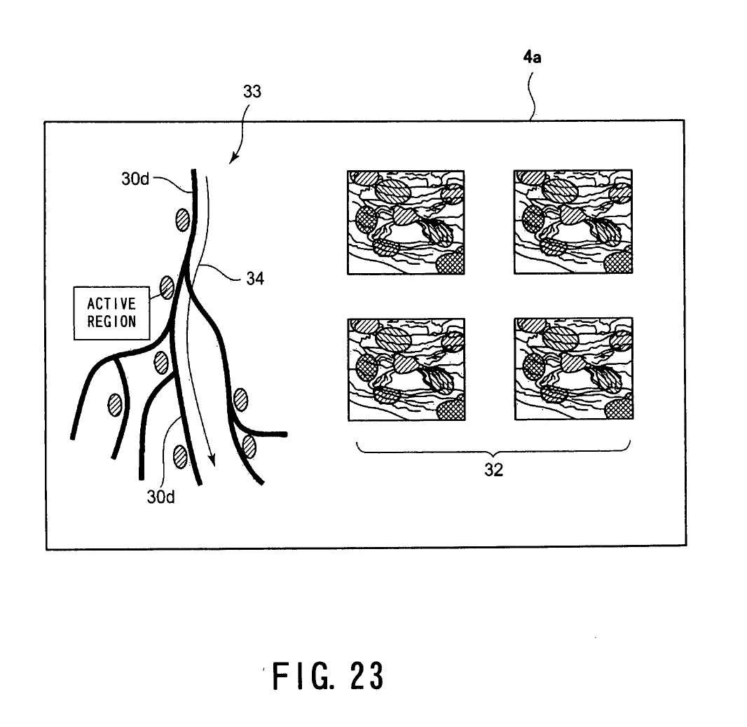

FIG. 23 is a drawing showing one example of the monitor screen of the display device 4.

Referring to FIG. 23, the display control unit 19 allows the monitor screen 4a of the display device 4 to simultaneously display the three-dimensional image indicating the appearance of a blood vessel structure 33 created by the processing in step S47 and a three-dimensional image 32 via the virtual endoscopy created by the processing in step S47. In this case, the three-dimensional image of the blood vessel structure 33 is created by the parallel-projection image creating section 18a or the perspective-projection image creating section 18b.

For example, the display control unit 19 allows the monitor screen 4a of the display device 4 to simultaneously display a plurality of pieces of the three-dimensional image data via the virtual endoscopy, serving as a plurality of the three-dimensional images 32, created along the path 30ae by the perspective-projection image creating section 18b. That is, the display control unit 19 does allow the display device 4, not to sequentially display the plurality of the three-dimensional images 32 via the virtual endoscopy, created along the path 30ae, but to simultaneously display them.

Upon simultaneously displaying the plurality of the three-dimensional images 32 via the virtual endoscopy, the display control unit 19 allows the monitor screen 4a of the display device 4 to thumbnail-display the plurality of the three-dimensional images 32 via the virtual endoscopy. Further, referring to FIG. 23, the display control unit 19 allows the display device 4 to display the image of the blood vessel structure 33 as well as the plurality of the three-dimensional images 32 via the virtual endoscopy. Thus, the same monitor screen 4a simultaneously displays the plurality of the three-dimensional images 32 via the virtual endoscopy and the image of the blood vessel structure 33, serving as the three-dimensional images indicating the appearance. Note that the display control unit 19 may allow the display device 4 to display only the plurality of the three-dimensional images 32 via the virtual endoscopy, without the image display operation of the blood vessel structure 33 on the display device 4.

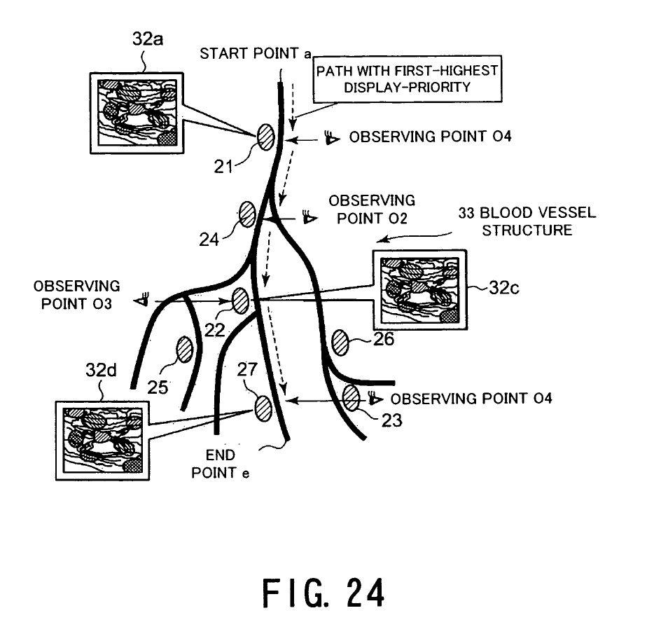

FIG. 24 is a drawing showing another example of the monitor screen of the display device 4.

Referring to FIG. 24, the display control unit 19 allows the three-dimensional image 32 via the virtual endoscopy created by the processing in step S47 to be displayed on the blood vessel 33, serving as the three-dimensional image indicating the appearance of the diagnostic portion, created by the processing in step S47. For example, the display control unit 19 allows the three-dimensional images 32 via the virtual endoscopy to be displayed with a balloon near the position of the active region on the blood vessel 33.

The blood vessel 33 shown in FIG. 24 is created on the basis of the first fused-image data, serving as the volume data created by the processing in step S44.

Specifically, the display control unit 19 allows three-dimensional images 32a, 32b, 32c, and 32d via virtual endoscopy to be displayed with a balloon near the position of the active region on the blood vessel structure 33. The display control unit 19 allows the three-dimensional image 32a via the virtual endoscopy, created at the observing point O1, to be displayed with a balloon near the position of the region 21, serving as the active region on the blood vessel structure 33, and the three-dimensional image 32b via the virtual endoscopy, created at the observing point O2 near the position of the region 24, serving as the active region on the blood vessel structure 33. Similarly, the three-dimensional images 32c and 32d via the virtual endoscopy at the observing point O3 and O4 are displayed with a balloon.

On the display screen shown in FIG. 24, the corresponding relationship between the blood vessel structure 33 and the three-dimensional images 32a, 32b, 32c, and 32d via the virtual endoscopy becomes obvious, when the point-of-view 400 is discretely moved and the three-dimensional images via the virtual endoscopy are displayed. Therefore, the display screen shown in FIG. 34 enables the efficient interpretation.

Further, the plurality of the three-dimensional images 32 via the virtual endoscopy are simultaneously displayed and diagnostic information can be sufficiently presented to a doctor and the like.

When the display device 4 simultaneously displays the plurality of the three-dimensional images 32 via the virtual endoscopy, similarly to the first and second embodiments, an operator selects the image and the display control unit 19 may allow the display device 4 to enlarge and display the selected three-dimensional images 32.

Further, referring to FIG. 23, the display control unit 19 may superimpose a marker 34 along the displayed path 30ae to the blood vessel structure 33 and may allow the display device 4 to display the superimposed marker 34 so as to distinguish the path 30ae of the currently displayed three-dimensional image 32 via the virtual endoscopy from another path. The marker 34 is displayed along the displayed path and a doctor can determine the path whose image is displayed via the virtual endoscopy on the blood vessel structure 33. Further, the display control unit 19 may the display device 4 to display a display color of the currently displayed path 30ae, which is different from a display color of another path. In accordance with the change from one path to another path to be currently displayed, the display control unit 19 changes the display colors of the changed paths so as to distinguish the display color of the currently displayed path from those of other paths. Thus, the currently displayed path can be determined.

The three-dimensional image data is created along the path 30ae with the first-highest display-priority, from the start point 30a to the end point 30e and the three-dimensional image is displayed. Subsequently, the image creating unit 18 creates the three-dimensional image data along the path with the second-highest display-priority, from the start point 30a to the end point 30e. Under the control of the display control unit 19, the display device 4 displays the three-dimensional image data via the virtual endoscopy along the path with the second-highest display-priority, serving as the three-dimensional image. When the display-priority determining unit 41 determines the path 30ad with the second-highest display-priority, similarly to the path 30ae, the image creating unit 18 creates the three-dimensional image data along the path 30ad, from the start point 30a to the end point 30d, the display device 4 displays the three-dimensional image data, serving as the three-dimensional image. Further, the three-dimensional image data is created along the path with the next-highest display-priority and the created three-dimensional image data is displayed.

The image creating unit 18 may create only the three-dimensional image data along the path with the highest display-priority, and the display control unit 19 may allow the display device 4 to display only the three-dimensional image data along the path with the highest display-priority.

The display control unit 19 may allow the display device 4 to display one path whose three-dimensional image data is created and displayed from the start point 30a to the end point 30e with the change of display color of the one path, different from that of another path, for the purpose of distinguishment from the other path.

Upon creating the three-dimensional image data along the path and displaying the created image data, as the three-dimensional image, the three-dimensional image data may be created by changing the line-of-sight direction for each active region. That is, similarly to the second embodiment, the three-dimensional image data viewed in the line-of-sight direction (e.g., direction A, B, or C shown in FIG. 16) varied depending on the active region may be created and the created image data may be displayed as the three-dimensional image. Thus, it is possible to observe the active region at the deepest position, which cannot be observed with the three-dimensional image created along the path.

When the diagnostic portion is moved, similarly to the first and second embodiments, the image creating unit 18 may create the three-dimensional image data by executing the volume rendering at the position with the constant distance between the point-of-view 400 and the active region by changing the position of the point-of-view 400 in accordance with the movement of the diagnostic portion. Further, the volume rendering may be executed by fixing the position of the point-of-view 400.

With the diagnostic imaging system 1B and the image processing system 3B according to the present invention, the display-priority is determined on the basis of the relationship between the path of the tubular region and the active region existing around the path, the superimposed image is created in accordance with the display-priority, and the created image is sequentially displayed, thereby displaying and observing the three-dimensional image along the path. Thus, it is possible to efficiently make a diagnosis and a diagnostic reading by the doctor or the like, because a time for searching the targeted active region by the doctor or the like can be reduced.

What is claimed is:

1. A diagnostic imaging system for creating an image of inside of a tubular tissue of an object on the basis of volume data obtained by capturing an image of the object and using the created image for diagnosis, the diagnostic imaging system comprising:
an active region extracting unit for obtaining functional information data indicating functional information of the object, and for extracting an active region from the functional information data;
an image data fusing unit for fusing the active region extracted by the active region extracting unit and the image of the inside of the tubular tissue; and
a display control unit for allowing the image fused by the image data fusing unit to be displayed.

2. A diagnostic imaging system according to claim 1, further comprising:
a display-priority determining unit for determining a display-priority for displaying a plurality of the active regions extracted by the active region extracting unit,
wherein the display control unit fuses the functional image of at least the active region with a highest display-priority determined by the display-priority determining unit and the image of the inside of the tubular tissue, and allows the fused image to be displayed.

3. A diagnostic imaging system according to claim 2, wherein the display control unit sequentially displays the images of the plurality of the active regions in accordance with the display-priority.

4. A diagnostic imaging system according to claim 2, wherein the display-priority determining unit determines the display-priority on the basis of a volume of the active region or a voxel value of the active region.

5. A diagnostic imaging system according to claim 3, wherein the display-priority determining unit determines the display-priority on the basis of a volume of the active region or a voxel value of the active region.

6. A diagnostic imaging system for creating an image of a tubular tissue of an object on the basis of volume data obtained by capturing an image of the object and using the created image for diagnosis, the diagnostic imaging system comprising:
an active region extracting unit for obtaining functional information data indicating functional information of the object, and for extracting an active region from the functional information data;
an image data fusing unit for fusing the active region extracted by the active region extracting unit and an image indicating a path of the tubular tissue; and
a display control unit for allowing the image fused by the image data fusing unit to be displayed.

7. A diagnostic imaging system according to claim 6, wherein the tubular tissue has a plurality of paths, and the display control unit fuses the active regions extracted by the active region extracting unit and the paths in a form that the active regions goes along the paths, and allows the fused image to be displayed.

8. A diagnostic imaging system according to claim 7, wherein the display control unit fuses a functional image of the active region and an image of the inside of the tubular tissue to create a thumbnail image, and allows the thumbnail image to be displayed along the path of the tubular tissue.

9. A diagnostic imaging system comprising:
an image data fusing unit for fusing functional image data, serving as volume data collected by capturing an object, and morphological image data, serving as the volume data, to create fused-image data, serving as the volume data;
an active region extracting unit for extracting the active region from the functional image data;
an image creating unit for creating three-dimensional image data obtained by superimposing the functional image and the morphological image along a specific line-of-sight direction relative to the active region, on the basis of the fused-image data; and
a display control unit for allowing the three-dimensional image data to be displayed as a three-dimensional image.

10. An image processing system for creating an image of inside of a tubular tissue of an object on the basis of volume data obtained by capturing an image of the object and using the created image for diagnosis, the image processing system comprising:
an active region extracting unit for obtaining functional information data indicating functional information of the object, and for extracting an active region from the functional information data;
an image data fusing unit for fusing the active region extracted by the active region extracting unit and the image of the inside of the tubular tissue; and
a display control unit for allowing the image fused by the image data fusing unit to be displayed.

11. An image processing system according to claim 10, further comprising:
a display-priority determining unit for determining a display-priority for displaying a plurality of the active regions extracted by the active region extracting unit,
wherein the display control unit fuses the functional image of at least the active region with a highest display-priority determined by the display-priority determining unit and the image of the inside of the tubular tissue, and allows the fused image to be displayed.

12. An image processing system according to claim 11, wherein the display control unit sequentially displays the images of the plurality of the active regions in accordance with the display-priority.

13. An image processing system according to claim 11, wherein the display-priority determining unit determines the display-priority on the basis of a volume of the active region or a voxel value of the active region.

14. An image processing system according to claim 12, wherein the display-priority determining unit determines the display-priority on the basis of a volume of the active region or a voxel value of the active region.

15. An image processing system for creating an image of a tubular tissue of an object on the basis of volume data obtained by capturing an image of the object and using the created image for diagnosis, the image processing system comprising:
an active region extracting unit for obtaining functional information data indicating functional information of the object, and for extracting an active region from the functional information data;
an image data fusing unit for fusing the active region extracted by the active region extracting unit and an image indicating a path of the tubular tissue; and
a display control unit for allowing the image fused by the image data fusing unit to be displayed.

16. An image processing system according to claim 15, wherein the tubular tissue has a plurality of paths, and the display control unit fuses the active regions extracted by the active region extracting unit and the paths in a form that the active regions goes along the paths, and allows the fused image to be displayed.

17. An image processing system according to claim 16, wherein the display control unit fuses a functional image of the active region and an image of the inside of the tubular tissue to create a thumbnail image, and allows the thumbnail image to be displayed along the path of the tubular tissue.

18. An image processing system comprising:
an image data fusing functional image data, serving as volume data collected by capturing an object, and morphological image data, serving as the volume data, to create fused-image data, serving as the volume data;
an active region extracting unit for extracting the active region from the functional image data;
an image creating unit for creating three-dimensional image data obtained by superimposing the functional image and the morphological image along a specific line-of-sight direction relative to the active region, on the basis of the fused-image data; and
a display control unit for allowing the three-dimensional image data to be displayed as a three-dimensional image.
